(19) United States
(12) Patent Application Publication
(10) Pub. No.: US 2021/0252104 A1
(43) Pub. Date: Aug. 19, 2021

(54) ANTIBACTERIAL PEPTIDE MONOMERS AND COMBINATIONS FOR CO-THERAPY

(71) Applicant: ARREVUS, INC., Butner, NC (US)

(72) Inventors: Carl N. KRAUS, Raleigh, NC (US); Laszlo OTVOS, Audubon, PA (US)

(21) Appl. No.: 17/259,301

(22) PCT Filed: Jul. 12, 2019
(86) PCT No.: PCT/US2019/041656

§ 371 Date: Jan. 11, 2021

Related U.S. Application Data
(60) Provisional application No. 62/697,019, filed on Jul. 12, 2018.
(60) Provisional application No. 62/777,897, filed on Dec. 11, 2018.

(51) Int. Cl. A61K 38/12; A61P 31/04; A61K 31/407

(52) U.S. Cl. CPC A61K 38/12; A61K 31/407; A61P 31/04

(57) ABSTRACT

Described herein are combinations of the antibacterial peptide monomers, and analogs, derivatives thereof, with antibiotics. The invention further relates to methods for using the combination to increase the sensitivity of antibiotic resistant bacteria to an antibiotic, widen the therapeutic index of the antibacterial peptide, and treat bacterial infections in a host animal.

### TECHNICAL FIELD

The invention described herein relates to treating bacterial infections with combinations of antibacterial peptide monomers with antibiotics. The invention also relates to methods for using the combinations to increase the sensitivity of antibiotic resistant bacteria to an antibiotic, widen the therapeutic index of the antibacterial peptides, and treat bacterial infections in a host animal.

### BACKGROUND

Antibiotic resistance of microorganisms is a growing dilemma worldwide. The overuse of currently available antibiotics has contributed to this issue. The development of new antibiotics and alternative methods of treating infections is needed.

For example, blood stream infections (BSIs), such as bacteremia, represent a serious and increasing clinical problem due to the high mortality and treatment failures because of high rates of antibiotic resistance. Bacteremia caused at least in part by Acinetobacter baumannii has an especially poor prognosis due to the fewer available treatment options for Gram-negative infection, and also because of the prevalence of multi-drug resistant (MDR) A. baumannii, such as the clinical HUMC1 strain. Treatment is typically empiric for patients at high risk of harboring MDR Gram-negative pathogens, and options are limited to carbapenems in combination with a quinolone or aminoglycoside, or so-called anti-pseudomonal β-lactams. Imipenem (IPM), a standard-of-care β-lactam antibiotic, has a broad spectrum of activity against aerobic and anaerobic, Gram-positive and Gram-negative bacteria, including many MDR strains (e.g., A. baumannii). However, many A. baumannii strains have developed resistance to IPM.

Similarly, nosocomial infections, such as hospital-acquired pneumonia (HAP or HABP) and ventilator-associated pneumonia (VAP) not only have high mortality and treatment failures, but also often include infections that are caused at least in part by resistant pathogens and bacteria.

There remains a need for other compounds and combinations of compounds that can lower the resistance of antibiotic-resistant microorganisms and treat antibiotic-resistant infections.

### SUMMARY OF THE INVENTION

It has been discovered that monomer peptides described herein, when administered with other antibiotics are highly effective against pathogenic infections, such as bacterial infections. It has also been unexpectedly discovered that monomer peptides are synergistic with other antibiotics.

In one embodiment, monomer peptides, such as Chex1-Arg20, also known as ARV-1502, and analogs, and derivatives thereof, and pharmaceutically acceptable salts of any of the foregoing are described.

In another embodiment, compositions comprising peptide monomers such as Chex1-Arg20, analogs and derivatives thereof, and pharmaceutically acceptable salts thereof in combination with one or more other antibiotics are described.

In another embodiment, uses of monomer peptides, such as Chex1-Arg20 and analogs, and derivatives thereof, and pharmaceutically acceptable salts of any of the foregoing, or compositions comprising any of the foregoing in methods for treating pathogenic infections, such as bacterial infections, and/or in the manufacture of medicaments for treating pathogenic infections in a host animal are described. The methods comprise contacting the bacteria with or administering one or more monomer peptides, or compositions thereof, and another antibiotic to the host animal.

In another embodiment, uses of compositions comprising monomer peptides, such as Chex1-Arg20 and analogs, and derivatives thereof, and pharmaceutically acceptable salts of any of the foregoing in combination with one or more other antibiotics in methods for treating pathogenic infections, such as bacterial infections, and/or in the manufacture of medicaments for treating pathogenic infections in a host animal are described. The methods comprise contacting the bacteria with or administering such compositions to the host animal.

In another embodiment, uses of monomer peptides or compositions comprising peptide monomers, such as Chex1-Arg20, analogs and derivatives thereof, and pharmaceutically acceptable salts thereof, or compositions comprising any of the foregoing in combination with other antibiotics where the peptide monomers and/or the antibiotics widen the therapeutic index of other antibiotics, and/or enhance the activity of other antibiotics and the overall treatment, and/or increase the sensitivity of resistant bacteria to the other antibiotics for treating pathogenic infections in a host animal are described. In another embodiment, uses of monomer peptides or compositions comprising peptide monomers, such as Chex1-Arg20, analogs and derivatives thereof, and pharmaceutically acceptable salts thereof, or compositions comprising any of the foregoing in combination with other antibiotics in the manufacture of medicaments for widening the therapeutic index of other antibiotics, and/or enhancing the activity of other antibiotics and the overall treatment, and/or increasing the sensitivity of resistant bacteria to the other antibiotics for treating pathogenic infections in a host animal are described.

In another embodiment, uses of monomer peptides, such as Chex1-Arg20 and analogs, and derivatives thereof, and pharmaceutically acceptable salts of any of the foregoing, or compositions comprising any of the foregoing in methods for treating resistant pathogenic infections, including bacteria resistant to penicillin and related compounds, and/or bacteria expressing extended-spectrum beta lactamases (ESBLs), and/or in the manufacture of medicaments for treating resistant pathogenic infections, including bacteria resistant to penicillin and related compounds, and/or bacteria expressing extended-spectrum beta lactamases (ESBLs) in a host animal are described. The methods comprise contacting the bacteria with or administering one or more monomer peptides, or compositions thereof, and another antibiotic to the host animal.

In another embodiment, uses of monomer peptides, such as Chex1-Arg20 and analogs, and derivatives thereof, and pharmaceutically acceptable salts of any of the foregoing, or compositions comprising any of the foregoing in methods for reducing the risk of recurrence or relapse of a pathogenic infection, such as a bacterial infection, and/or in the manufacture of medicaments for reducing the risk of recurrence or relapse of a pathogenic infection in a host animal are described. The methods comprise administering one or more monomer peptides, or compositions thereof, and another antibiotic to the host animal.

In another embodiment, uses of monomer peptides, such as Chex1-Arg20 and analogs, and derivatives thereof, and pharmaceutically acceptable salts of any of the foregoing, or compositions comprising any of the foregoing in methods for increasing the sensitivity of antibiotic resistant bacteria to an antibiotic, and/or in the manufacture of medicaments for increasing the sensitivity of antibiotic resistant bacteria to an antibiotic are described. The methods comprise administering one or more monomer peptides, or compositions thereof to the host animal, and administering another antibiotic to the host animal.

It has been unexpectedly discovered that the monomer peptides described herein significantly enhance the activity of other antibiotics, leading to wider therapeutic indices, increased bacterial sensitivity, including increasing sensitivity of resistant bacteria that may try to survive treatment by the conventional antibiotic with various defense mechanisms, and lower recurrence and/or relapse rates, which might arise from infection recovery by surviving bacteria. Without being bound by theory, it is believed herein that such monomer peptides, though they may not have sufficient activity as a monotherapy, are capable of taking advantage of the distress caused by the co-administered antibiotic. That capability is not believed to be limited to the specific modes of action of the co-administered antibiotic. Instead, the effects of the monomer peptides are general in co-therapy protocols where the bacteria have been placed under stress conditions by administration of the other antibiotic.

### BRIEF DESCRIPTION OF THE DRAWINGS

FIGS. 1A-1C show A. baumannii burden in the target tissues kidney (1A), spleen (1B), and liver (1C) quantified as CFU per gram of target tissues in the murine bacteremia model following different treatment groups. *P<0.001 vs control and IPM/CIL alone groups, **P<0.05 vs respective ARV-1502 monotherapy group, using Student's t test. At least six mice per group were analyzed.

FIG. 2 shows percent of negative cultures in blood in the murine bacteremia model following different treatment groups.

FIG. 3 shows synergy between peptide Chex1-Arg20 and meropenem against the carbapenem-resistant Escherichia coli UNT167-1 (CRE) strain in vitro. The shaded boxes indicate no visible signs of bacterial growth; the plus signs indicate visually visible bacterial growth in the wells. The antibiotics and the peptides were applied to bacteria in mid-log growing phase concomitantly.

### DETAILED DESCRIPTION

In one embodiment, the monomer peptide is selected from the group consisting of
R¹-AAₙ-Pro-Arg-Pro-AAₘ-R²
R¹-AAₙ-Pro-Arg-Pro-Pro-AAₘ-R²
R¹-AAₙ-Pro-Arg-Pro-Pro-Arg-AAₘ-R²
R¹-AAₙ-Pro-Arg-Pro-Pro-Arg-Pro-AAₘ-R²
R¹-AAₙ-Arg-Pro-Pro-Arg-Pro-AAₘ-R²
R¹-AAₙ-Pro-Pro-Arg-Pro-AAₘ-R²
   or a salt thereof, where R¹ is H or an acyl group, R² is NH₂, NHNH₂, OH; each AA is an independently selected amino acid, n is an integer in the range from 1 to 10, and m is an integer in the range from 1 to 10.

In another embodiment, the monomer peptide is of the formula

X¹—X²-Pro-X³—X⁴-Pro-Arg-Pro-Tyr-Leu-Pro-X⁵-Pro-Arg-Pro-Pro-Arg-Pro-Y

or a salt thereof, wherein X¹ is a natural or non-natural amino acid having a free amino group or 1-amino-cyclohexyl carboxyl (“Chex”), X² is Arg or N-methyl-Arg, X³ is Asp or GIu, X⁴ is Arg or Lys, X⁵ is Arg or Lys, and Y is Arg, Arg-NH₂, N-methyl-Arg, N-methyl-Arg-NH₂, Val-Arg, Val-Arg-NH₂, Val-(N-methyl-Arg), or Val-(N-methyl)-Arg-NH₂, including terminal OH and NHNH₂ analogs, and salts of any of the foregoing.

In another embodiment, the monomer peptide is selected from the group consisting of
Xaa-Arg-Pro-Asp-Lys-Pro-Arg-Pro-Tyr-Leu-Pro-Arg-Pro-Arg-Pro-Pro-Arg-Pro-Val-Arg
Xaa-Arg-Pro-Asp-Lys-Pro-Arg-Pro-Tyr-Leu-Pro-Arg-Pro-Arg-Pro-Pro-Arg-Pro-Val
Xaa-Arg-Pro-Asp-Lys-Pro-Arg-Pro-Tyr-Leu-Pro-Arg-Pro-Arg-Pro-Pro-Arg-Pro-Val-Arg
Arg-Pro-Tyr-Leu-Pro-Arg-Pro-Arg-Pro-Pro-Arg-Pro-Val-Arg
Asp-Lys-Gly-Ser-Tyr-Leu-Pro-Arg-Pro-Thr-Pro-Pro-Arg-Pro-Ile-Tyr-Asn-Arg
Xaa-Xaa-Pro-Xaa-Xaa-Pro-Arg-Pro-Tyr-Leu-Pro-Xaa-Pro-Arg-Pro-Pro-Arg-Pro-Xaa-Xaa
Xaa-Arg-Pro-Asp-Lys-Pro-Arg-Pro-Tyr-Leu-Pro-Arg-Pro-Arg-Pro-Pro-Arg-Pro-Val-Arg-Xaa
   or a salt thereof, where Xaa is in each instance an independently selected amino acid. In addition, in each of the foregoing monomers, the N-terminus may be acylated, and the C-terminus may include OH, NH₂, NHNH₂, and the like.

In another embodiment, the monomer peptide is selected from the group consisting of

| Example | Peptide Monomer | Chex1-Arg20 analog |
|---|---|---|
| 1 | Chex-RPDKPRPYLPRPRPPRPVR-NH2 | monomer |
| (Chex1-Arg20) | | |
| 2 | Ac-RPDKPRPYLPRPRPPRPVR-NH2 | Ac-monomer |
| 3 | Bu-RPDKPRPYLPRPRPPRPVR-NH2 | Bu-monomer |
| 4 | Va-RPDKPRPYLPRPRPPRPVR-NH2 | Va-monomer |
| 5 | Gu-RPDKPRPYLPRPRPPRPVR-NH2 | Gu-monomer |
| 6 | Chex-RPDKPRPYLPRPRPPRPVR-OH | monomer-acid |
| 7 | Chex-RPDKPRPYLPRPRPPRPVR-NH-NH2 | monomer-hydrazide |
| 8 | Chex-RPDKPRPYLPRPRPPRPVR-OH | monomer-alcohol |

where Ac=acetyl-NH, Bu=butyryl-NH, Va=valeryl-NH, Gu=N,N,N′,N′-tetramethylguanidinyl-NH. It is to be understood that salts of the foregoing Examples are also described herein.

In another embodiment, the monomer peptide is selected from the group consisting of
Chex-Arg-Pro-Asp-Lys-Pro-Arg-Pro-Tyr-Leu-Pro-Arg-Pro-Arg-Pro-Pro-Arg-Pro-Val-Arg Chex-Arg-Pro-Asp-Lys-Pro-Arg-Pro-Tyr-Leu-Pro-Arg-Pro-Arg-Pro-Pro-Arg-Pro-Val-Arg-NH₂ or a salt thereof.

In another embodiment, the monomer peptide is Chex1-Arg20, or an analog of derivative thereof, or a salt thereof.

Additional illustrative, but nonlimiting, examples of monomer peptides, and analogs and derivatives thereof, are described in U.S. Pat. No. 8,492,515, the disclosure of which is incorporated herein by reference in its entirety.

Additional illustrative, but nonlimiting, examples of monomer peptides, and analogs and derivatives thereof, are described in Li et al., “C-Terminal Modifications Broaden Activity of the Proline-Rich Antimicrobial Peptide, Chex1-Arg20,” Aust J Chem 68:1373-78 (2015), the disclosure of which is incorporated herein by reference in its entirety.

In another embodiment, the monomer peptide is Chex1-Arg20, or a salt thereof.

In another embodiment, methods are described herein for treating pathogenic infections in host animals where a therapeutically effective amount of one or more of monomer peptides, such as Chex1-Arg20, and analogs, and derivatives thereof, and pharmaceutically acceptable salts of any of the foregoing are administered to the host animals, and/or are used in the manufacture of a medicament for administering to the host animals in conjunction with a therapeutically effective amount of one or more other antibiotics

In each of the foregoing methods, it has been discovered that using the monomer peptides described herein in combination with other antibiotics, leads to fewer toxicity-related adverse events in the host animal. It has been reported that conventional therapies, using for example penicillin and related compounds, often results in a high number of toxicity-related adverse events. For example, it has been discovered that the monomer peptides described herein are able to pass through the bacterial membrane to reach the target cell. Many conventional antimicrobial compounds enter pathogenic bacteria by disrupting the bacterial membrane. That same mechanism of disrupting bacterial membranes is reportedly responsible, at least in part, for the off-target toxicity and/or other adverse events of effects of conventional antimicrobial compounds. Co-administering monomer peptides provides a method for administering other peptides at lower doses and resulting in fewer toxicity-related adverse event.

Additional illustrative embodiments of the invention are described by the following non-limiting clauses:

A method for treating a bacterial infection in a host animal, the method comprising administering (a) one or more monomer peptides described herein and (b) one or more antibiotics to the host animal.

The method of the preceding clause where the infection is a bloodstream infection (BSI), such as bacteremia.

The method of any one of the preceding clauses where the infection is a urinary tract infection (UTI), such as a nosocomial UTI, and including complicated UTIs.

The method of any one of the preceding clauses where the infection is pneumonia, such a community-acquired bacterial pneumonia (CAP or CABP), nosocomial pneumonia or hospital-acquired pneumonia (HAP or HABP), ventilator-associated pneumonia (VAP), and the like.

The method of any one of the preceding clauses where the infection is associated with a medical procedure, such as a surgery.

The method of any one of the preceding clauses where the infection is peritonitis.

The method of any one of the preceding clauses where the infection is caused at least in part by Gram-negative bacteria.

The method of any one of the preceding clauses where the infection is caused at least in part by Gram-negative bacilli.

The method of any one of the preceding clauses where the infection is caused at least in part by Mycoplasma.

The method of any one of the preceding clauses where the infection is caused at least in part by one or more pathogenic organisms that are resistant to one or more antibiotics.

The method of any one of the preceding clauses where the infection is caused at least in part by one or more extended spectrum beta-lactamase (ESBL) bacteria.

The method of any one of the preceding clauses where the infection is caused at least in part by one or more carbapenemase producing bacteria, such as KPC-producing bacteria and the like.

The method of any one of the preceding clauses where the infection is caused at least in part by Acinetobacter.

The method of any one of the preceding clauses where the infection is caused at least in part by Acinetobacter baumannii.

The method of any one of the preceding clauses where the A. baumannii includes resistant A. baumannii.

The method of any one of the preceding clauses where ere the A. baumannii includes A. baumannii resistant to imipenem or cilastatin, or both. The method of any one of the preceding clauses where the A. baumannii includes the HUNC1 strain.

The method of any one of the preceding clauses where the infection is caused at least in part by Enterobacteriaceae.

The method of any one of the preceding clauses where the Enterobacteriaceae includes resistant Enterobacteriaceae.

The method of any one of the preceding clauses where the Enterobacteriaceae includes carbapenem-resistant Enterobacteriaceae (CRE) and/or carbapenemase-producing Enterobacteriaceae (CPE).

The method of any one of the preceding clauses where the infection is caused at least in part by Staphylococcus, such as S. aureus.

The method of any one of the preceding clauses where the S. aureus includes resistant S. aureus, such as MRSA, VISA, VRSA, and the like.

The method of any one of the preceding clauses where the infection is caused at least in part by Escherichia coli.

The method of any one of the preceding clauses where the E. coli includes resistant E. coli.

The method of any one of the preceding clauses where the infection is caused at least in part by Klebsiella, such as K. pneumoniae.

The method of any one of the preceding clauses where the K. pneumoniae includes resistant K. pneumoniae.

The method of any one of the preceding clauses where the infection is caused at least in part by carbapenemase producing K. pneumoniae.

The method of any one of the preceding clauses where the infection is caused at least in part by Legionella.

The method of any one of the preceding clauses where the infection is caused at least in part by Pseudomonas, such as P. aeruginosa.

The method of any one of the preceding clauses where the infection is caused at least in part by resistant P. aeruginosa, such as mucoidal P. aeruginosa.

The method of any one of the preceding clauses where the monomer peptide is a DnaK inhibitor.

The method of any one of the preceding clauses where the monomer peptide is a host defense peptide (HDP).

The method of any one of the preceding clauses where the monomer peptide is Chex1-Arg20.

The method of any one of the preceding clauses where the one or more monomer peptides are administered with one or more beta-lactam antibiotics, such as penicillins, penams, carbapenems, oxapenams, penems, carbapenems, monobactams, cephems, carbacephems, oxacephems, and the like.

The method of any one of the preceding clauses where the one or more monomer peptides are administered with one or more penicillins, including ureidopenicillins, such as piperacillin, and the like.

The method of any one of the preceding clauses where the one or more monomer peptides are administered with one or more carbapenems, such as imipenem, combinations of imipenem and cilastatin, and the like.

The method of any one of the preceding clauses where the one or more monomer peptides are administered with one or more tetracyclines, such as tigecycline, and the like.

The method of any one of the preceding clauses where the one or more monomer peptides are administered with one or more polypeptide antibiotics or polymyxins, such as colistin, and the like.

The method of any one of the preceding clauses where the one or more monomer peptides are administered with one or more quinolone antibiotics.

The method of any one of the preceding clauses where the one or more monomer peptides are administered with one or more aminoglycoside antibiotics.

The method of any one of the preceding clauses where the one or more monomer peptides are administered with one or more antipseudomonal beta lactam antibiotics, such as sulbactam, ampicillin/sulbactam, and the like.

The method of any one of the preceding clauses where the one or more monomer peptides are parenterally administered.

The method of any one of the preceding clauses where the one or more monomer peptides are administered by intravenous (iv) injection.

The method of any one of the preceding clauses where the one or more monomer peptides are administered by intramuscular (im) injection.

The method of any one of the preceding clauses where the one or more monomer peptides and the antibiotic are administered simultaneously.

The method of any one of the preceding clauses where the one or more monomer peptides and the antibiotic are administered sequentially.

The method of any one of the preceding clauses where the one or more monomer peptides and the antibiotic are administered sequentially, where the antibiotic is administered after a predetermined time following the administration of the one or more monomer peptides.

The method of any one of the preceding clauses where the predetermined time is between about 30 minutes and about 12 hours, or between about 30 minutes and about 8 hours, between about 30 minutes and about 6 hours, between about 30 minutes and about 4 hours.

The method of any one of the preceding clauses where the predetermined time is between about 1 and about 12 hours, or between about 1 and about 8 hours, between about 1 and about 6 hours, between about 1 and about 4 hours.

In another embodiment, the compounds and compositions described herein are used in the manufacture of one or more medicaments that are capable of being used in or adapted for use in any of the methods described herein.

It is to be understood that the therapeutically effective amount of the one or more peptide monomers and the therapeutically effective amount of the one or more antibiotics refers to those amounts that are used in the combination and co-therapy methods described herein. Therefore, such therapeutically effective amounts will be understood to often include amounts of each component that might not be optimal and/or therapeutically effective if it were used in a monotherapy, or alternative therapy that did not include at least one peptide monomer and at least one antibiotic.

In each of the foregoing methods, it is to be understood that the methods for treating infections may be used in conjunction with other treatments and procedures, including but not limited to treating cancer, organ transplantation, companion therapy to joint replacements, corneal disease treatment, and the like.

In reciting the foregoing clauses and embodiments, and also including the embodiments following, it is to be understood that all possible combinations of features, and all possible subgenera and sub-combinations are described.

In each of the foregoing and in each of the following embodiments, unless otherwise indicated, it is to be understood that the compounds included in the compositions, methods and uses include and represent not only all pharmaceutically acceptable salts of the compounds, but also include any and all hydrates and/or solvates of the compound formulae. It is appreciated that certain functional groups, such as the hydroxy, amino, and like groups form complexes and/or coordination compounds with water and/or various solvents, in the various physical forms of the compounds. Accordingly, the above formulae are to be understood to be a description of such hydrates and/or solvates, including pharmaceutically acceptable solvates.

In each of the foregoing and in each of the following embodiments, unless otherwise indicated, it is also to be understood that the compounds included in the compositions, methods and uses include and represent any and all crystalline forms, partially crystalline forms, and non-crystalline and/or amorphous forms of the compounds.

In each of the foregoing and in each of the following embodiments, unless otherwise indicated, it is also to be understood that the compounds included in the compositions, methods and uses include and represent each possible isomer, such as stereoisomers and geometric isomers, both individually and in any and all possible mixtures.

Unless otherwise defined, all technical and scientific terms used herein have the same meaning as commonly understood by one of skill in the art to which this invention belongs. The terminology used in the description of the invention herein is for the purpose of describing certain embodiments herein, and is not intended to be limiting of the invention. All publications, patent applications, patents, patent publications and other references cited herein are incorporated by reference in their entireties for the teachings relevant to the sentence and/or paragraph and/or topic in which the reference is presented.

As used in the description of the invention and the appended claims, the singular forms “a,” “an,” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±10%, ±5%, ±1%, ±0.5%, or even ±0.1% of the specified amount.

The transitional phrase “consisting essentially of” means that the scope of a claim is to be interpreted to encompass the specified materials or steps recited in the claim, and those that do not materially affect the basic and novel characteristics of the claimed invention.

The term “modulate,” “modulates,” or “modulation” refers to enhancement (e.g., an increase) or inhibition (e.g., a decrease) in the specified level or activity.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In another embodiment, the inhibition or reduction results in little or essentially no detectible activity (at most, an insignificant amount, e.g., less than about 10% or even 5%). With reference to reduced dosing that is possible using the co-administration methods described herein, such reduced dosing is also illustratively reduced by 10%, 15%, 20%, 25%, 30%, 35%, 40%, 50%, 60%, or 75%.

A “therapeutically effective” amount as used herein is an amount that provides some improvement or benefit to the host animal. Alternatively stated, a “therapeutically effective” amount is an amount that will provide some alleviation, mitigation, or decrease in at least one clinical symptom in the host animal. Those skilled in the art will appreciate that the therapeutic effects need not be complete or curative, as long as some benefit is provided to the host animal.

By the terms “treat,” “treating,” or “treatment of” it is intended that the severity of the host animal's condition is reduced or at least partially improved or modified and that some alleviation, mitigation or decrease in at least one clinical symptom is achieved.

A “synergistic” effect, as used herein, is an effect that is greater than additive when two molecules are administered to a host animal simultaneously or sequentially. It is to be understood that sequential administration may be in any order. It is also to be understood that sequential administration may include variations where a time delay between the co-administration steps is included within the co-administration dosing window.

The term “therapeutic index,” as used herein, refers to the ratio of the dose of drug that causes adverse effects at an incidence/severity not compatible with the targeted indication (e.g., toxic dose in 50% of subjects, TD50) to the dose that leads to the desired pharmacological effect (e.g., efficacious dose in 50% of subjects, ED50). A widening of the therapeutic index refers to an increase in the difference between the toxic and therapeutic dose.

The term “minimum inhibitory concentration (MIC)” refers to the lowest concentration of a compound or molecule that prevents visible growth of a bacterium.

The term “resistant,” as used herein, refers to the ability of a microorganism to resist the toxic effects of an antibiotic. Resistance typically occurs when the microorganism produces one or proteins or other components that can disable an antibiotic or prevents transport of the antibiotic into the cell. As used herein, the term also includes microorganisms that undergo reversal of tolerance. A resistant microorganism is one in which the minimum inhibitory concentration (MIC) is increase by at least 10% relative to the average MIC of the non-resistant strain.

The term “sequentially” refers to the administration of two or more agents one after the other and close enough in time that each of the agents exerts a biological activity on the other agent, e.g., the two or more agents have an effect in combination.

As used herein, the term “acyl” generally refers to the group R—C(O), where R is alkyl, cycloalkyl, heteroalkyl, cycloheteroalkyl, aryl, arylalkyl, heteroaryl, or heteroarylalkyl, each of which is optionally substituted.

As used herein, the term “amino acid” generally refers to an organic compound containing both a basic amino group and an acidic carboxyl group. Included within this term are natural amino acids (e.g., L-amino acids), modified and unusual amino acids (e.g., D-amino acids), as well as amino acids which are known to occur biologically in free or combined form but usually do not occur in proteins. Included within this term are modified and unusual amino acids, such as those disclosed in, for example, Roberts and Vellaccio (1983) The Peptides, 5: 342-429, the teaching of which is hereby incorporated by reference. Natural protein occurring amino acids include, but are not limited to, alanine, arginine, asparagine, aspartic acid, cysteine, glutamic acid, glutamine, glycine, histidine, isoleucine, leucine, lysine, methionine, phenylalanine, serine, threonine, tyrosine, tyrosine, tryptophan, proline, and valine. Natural non-protein amino acids include, but are not limited to arginosuccinic acid, citrulline, cysteine sulfinic acid, 3,4-dihydroxyphenylalanine, homocysteine, homoserine, ornithine, 3-monoiodotyrosine, 3,5-diiodotryosine, 3,5,5′-triiodothyronine, and 3,3′,5,5′-tetraiodothyronine. Modified or unusual amino acids which can be used to practice the invention include, but are not limited to, D-amino acids, hydroxylysine, 4-hydroxyproline, an N-Cbz-protected amino acid, 2,4-diaminobutyric acid, homoarginine, N-methyl-arginine, norleucine, N-methylaminobutyric acid, naphthylalanine, phenylglycine, beta-phenylproline, tert-leucine, 4-aminocyclohexylalanine, N-methyl-norleucine, 3,4-dehydroproline, N,N-dimethylaminoglycine, N-methylaminoglycine, 4-aminopiperidine-4-carboxylic acid, 6-aminocaproic acid, trans-4-(aminomethyl)-cyclohexanecarboxylic acid, 2-, 3-, and 4-(aminomethyl)-benzoic acid, 1-aminocyclopentanecarboxylic acid, 1-aminocyclopropanecarboxylic acid, and 2-benzyl-5-aminopentanoic acid.

Naturally occurring amino acids are represented herein in the manner recommended by the IUPAC-IUB Biochemical Nomenclature Commission, or (for amino acids) by either the one-letter code, or the three letter code, both in accordance with 37 C.F.R. § 1.822 and established usage.

The present invention is based in part on the ability of Chex1-Arg20, which has antibiotic activity, to enhance the activity of other antibiotics when administered in combination.

Additionally, the combination widens the therapeutic index for Chex1-Arg20. In another embodiment, the effectiveness of the combination may be synergistic compared to either compound alone. Furthermore, Chex1-Arg20 may be effective to increase the antibiotic sensitivity of antibiotic-resistant bacteria.

The bacterial target of peptide monomers described herein, such as Chex1-Arg20, is believed to be the C-terminal D-E helix of the 70 kDa bacterial heat shock protein DnaK (Otvos et al., J. Med. Chem. 48:5349 (2005); Kragol et al., Biochemistry 40:3016 (2001); Bikker et al., Chem. Biol. Drug Des. 68:148 (2006)). DnaK is a 650-residue protein comprised of 3 distinct domains: (1) N-terminal nucleotide binding domain, (2) C-terminal substrate binding domain and (3) flexible linking domain. Without being bound by theory, it is believed herein that peptide monomers described herein bind to DnaK, and show in vitro activity against multiple Gram-negative bacteria demonstrating antibacterial potency. For example, Chex1-Arg20 binds to to DnaK with a K_{d} of 0.41 (±0.01) μM. Binding of peptide monomers to DnaK inhibits opening and closing of the multi-helical lid over the peptide-binding pocket of DnaK and also inhibit the DnaK-mediated phosphate release from ATP. Peptide monomers described herein bind to DnaK from multiple bacterial species. The MPC of Chex1-Arg20 is reported to be >100 μM, supporting the conclusion that the antibacterial efficacy is not non-membranolytic. Because DnaK is present in all bacteria, peptide monomers described herein are effective against a wide variety of bacteria and may be effective in combination with all classes of antibiotics.

The peptide chains of Chex1-Arg20 contain acidic or basic groups (such as amine or carboxyl groups) and therefore it is to be understood that such groups can be optionally in the free base form or a salt form. All references to Chex1-Arg20, and analogs and derivatives thereof, unless indicated otherwise, also refer to various salt forms of the molecule, such as pharmaceutically acceptable salts.

The term “salts” includes addition salts of free acids or free bases. The term “pharmaceutically acceptable salt” refers to salts which possess toxicity profiles within a range that affords utility in pharmaceutical applications. Pharmaceutically unacceptable salts may nonetheless possess properties such as high crystallinity, which have utility in the compositions, methods and uses described herein, such as for example utility in process of synthesis, purification or formulation of compounds.

Suitable pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, .beta.-hydroxybutyric, salicylic, galactaric and galacturonic acid. Examples of pharmaceutically unacceptable acid addition salts include, for example, perchlorates and tetrafluoroborates.

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts. All of these salts may be prepared from Chex1-Arg20, and analogs and derivatives thereof, by reacting, for example, the appropriate acid or base with Chex1-Arg20, or analogs or derivatives thereof.

In another embodiment, compositions comprising Chex1-Arg20 or analogs and derivatives thereof, or a pharmaceutically acceptable salt thereof and an antibiotic are described. The compositions may comprise more than one antibiotic, e.g., 1, 2, 3, 4, or 5 or more antibiotics.

The composition may be a dosage form, e.g., a unit dosage form. In another embodiment, the monomer peptide, such a sChex1-Arg20 or analogs and derivatives thereof, or a pharmaceutically acceptable salt thereof and antibiotic are both present in therapeutically effective amounts. In another embodiment, the monomer peptide and antibiotic are both present in synergistic amounts, e.g., amounts that, when administered to a host animal, will produce a synergistic effect. It is to be understood that such amounts may be reduced, as defined herein, from those that would be used and/or required in any corresponding monotherapy.

In another embodiment, the peptide monomer, such as Chex1-Arg20 or analogs and derivatives thereof, or a pharmaceutically acceptable salt thereof and/or the antibiotic is present in an amount that, by itself, is not therapeutic but produces a therapeutic effect in combination. In another embodiment, the Chex1-Arg20 or analogs and derivatives thereof, or a pharmaceutically acceptable salt thereof and/or the antibiotic is present in an amount that, by itself, is not therapeutic but renders a third antibiotic therapeutically effective.

In another embodiment, the relative amount of the peptide monomer, such as Chex1-Arg20 or analogs and derivatives thereof, or a pharmaceutically acceptable salt thereof is minimized. It has been unexpectedly observed that excessively high doses of the Chex1-Arg20 or analogs and derivatives thereof, or a pharmaceutically acceptable salt thereof either do not provide a dose-response related improvement in outcome over the antibiotic alone and lower doses of the Chex1-Arg20 or analogs and derivatives thereof, or a pharmaceutically acceptable salt thereof. Accordingly, in another embodiment, the dose of the Chex1-Arg20 or analogs and derivatives thereof, or a pharmaceutically acceptable salt thereof is below a pre-determined threshold, and/or within the hermetic zone or range, as described herein. Without being bound by theory, it is believed herein that when efficacy is observed cease increasing with increasing dose, or plateau, the pharmacokinetics and/or pharmacodynamics of the corresponding therapies described herein may have reached a saturation point where there is a competing rate-limiting event, such as absorption, uptake, receptor cycling, and the like.

In another embodiment, a co-therapy is described where including one or more monomer peptides, such as Chex1-Arg20 or analogs and derivatives thereof, or a pharmaceutically acceptable salt thereof provides an expanded therapeutic window for the antibiotic. For example, at certain doses, colistin is reportedly nephrotoxic. The compositions, uses and methods described herein may increase the therapeutic window for colistin and other antibiotics providing for lower dosing protocols of colistin, where the administered dose of colistin and other antibiotics are reduced as defined herein, while maintaining therapeutic efficacy.

In another embodiment, a method of increasing the sensitivity of antibiotic resistant microorganisms, e.g., bacteria, to an antibiotic is described, where the method comprises contacting the bacteria with one or more monomer peptides, such as Chex1-Arg20 or analogs and derivatives thereof, or a pharmaceutically acceptable salt thereof. The method further comprises contacting the bacteria with the antibiotic or an alternative antibiotic.

In another embodiment, compositions, uses and methods for widening the therapeutic index of an antibiotic are described that include monomer peptides, such as Chex1-Arg20 or analogs and derivatives thereof, for treatment of a microorganism, e.g., bacteria, infection in a host animal, comprising administering to the host animal a therapeutically effective dose of Chex1-Arg20 or analogs and derivatives thereof, or a pharmaceutically acceptable salt thereof and an antibiotic. In another embodiment, the therapeutic index is widened by at least about 5%, e.g., at least about 10%, 20%, 50%, 100%, 200%, 500%, or more.

In another embodiment, compositions, uses and methods for reducing the risk of recurrence of a pathogenic microorganism, or bacterial, infection in a host animal are described, including administering to the host animal having an infection a therapeutically effective dose of Chex1-Arg20 or analogs and derivatives thereof, or a pharmaceutically acceptable salt thereof and an antibiotic.

In each of the uses and methods described herein, the one or more monomer peptides and one or more antibiotics may be administered simultaneously, including where the one or more monomer peptides and one or more antibiotics are in the same dosage form or in separate dosage forms, contemporaneously, or according to a protocol or dosing schedule where the one or more monomer peptides and one or more antibiotics are administered separately, and optionally after a predetermined lapse of time. It has been unexpectedly discovered that pre-administration of one or more monomer peptides following by an optional waiting period before administration of the one or more antibiotics provides better clinical and therapeutic outcomes.

Monomer peptides described herein, including Chex1-Arg20 or analogs and derivatives thereof, or a pharmaceutically acceptable salt thereof have been discovered to exhibit an unexpectedly long post-antibiotic effect (PAE). Thus, the uses and methods described herein can include dosing protocols and schedules where a delay of about 1, 2, 3, 4, 5, 6, 7, 8, 9, 10, 15, 20, 25, 30, 35, 40, 45, 50, 55, or 60 minutes or about 1.5, 2, 3, 4, 5, 6, 7, 8, 9, 10, 11, or 12 hours after either the monomer peptides or the antibiotics are administered. In another embodiment, the Chex1-Arg20 or analogs and derivatives thereof, or a pharmaceutically acceptable salt thereof is administered before the antibiotic. In other embodiments, the antibiotic is administered before the Chex1-Arg20 or analogs and derivatives thereof, or a pharmaceutically acceptable salt thereof.

In another embodiment, the host animal is one that has been diagnosed as being infected with antibiotic-resistant pathogenic organisms or is suspected of being infected with antibiotic-resistant pathogenic organisms. The host animal may not have been previously treated for the infection. The host animal may be administered Chex1-Arg20 or analogs and derivatives thereof, or a pharmaceutically acceptable salt thereof first to prime the host animal for antibiotic treatment.

In another embodiment, the host animal has been treated with an antibiotic and the treatment has been ineffective (e.g., the infection has not been reduced, has been reduced but not eradicated, or was thought to have been eradicated but has returned). Chex1-Arg20 or analogs and derivatives thereof, or a pharmaceutically acceptable salt thereof may be added to the treatment with the same antibiotic or with a different antibiotic.

In another embodiment, the host animal may be immunocompromised or otherwise have diminished ability to fight the infection.

The compositions, uses and methods described herein may permit the Chex1-Arg20 or analogs and derivatives thereof, or a pharmaceutically acceptable salt thereof and/or the antibiotic to be administered at a dose that that would not be therapeutically effective if administered alone. The MIC of the Chex1-Arg20 and analogs, and derivatives thereof, and/or antibiotic when provided together may be decreased by at least about 5%, e.g., at least about 10%, 15%, 20%, 25%, 30%, 35%, 40%, 45%, 50%, or more.

In another embodiment, the compositions, uses and methods described herein include treating an infection caused at least in part by one or more bacteria such as Rickettsia, Chlamydia, Mycobacteria, Clostridia, Corynebacteria, Mycoplasma, Ureaplasma, Legionella, Shigella, Salmonella, pathogenic Escherichia coli species, Bordatella, Neisseria, Treponema, Bacillus, Haemophilus, Moraxella, Vibrio, Staphylococcus spp., Streptococcus spp., Campylobacter spp., Borrelia spp., Leptospira spp., Erlichia spp., Klebsiella spp., Pseudomonas spp., Helicobacter spp., and any other pathogenic microorganism now known or later identified (see, e.g., Microbiology, Davis et al, Eds., 4^{th} ed., Lippincott, New York, 1990, the entire contents of which are incorporated herein by reference for the teachings of pathogenic microorganisms).

Illustrative examples of pathogenic organisms include, but are not limited to, Helicobacter pylori, Chlamydia pneumoniae, Chlamydia trachomatis, Ureaplasma urealyticum, Mycoplasma pneumoniae, Staphylococcus aureus, Streptococcus pyogenes, Streptococcus pneumoniae, Streptococcus viridans, Enterococcus faecalis, Neisseria meningitidis, Neisseria gonorrhoeae, Treponema pallidum, Bacillus anthracis, Salmonella typhi, Vibrio cholera, Pasteurella pestis (Yersinia pestis), Pseudomonas aeruginosa, Campylobacter jejuni, Clostridium difficile, Clostridium botulinum, Mycobacterium tuberculosis, Borrelia burgdorferi, Haemophilus ducreyi, Corynebacterium diphtheria, Bordetella pertussis, Bordetella parapertussis, Bordetella bronchiseptica, Haemophilus influenza, and enterotoxic Escherichia coli.

In another embodiment, the compositions, uses and methods described herein include treating an infection caused at least in part by one or more resistant bacteria such as Rickettsia, Chlamydia, Mycobacteria, Clostridia, Corynebacteria, Mycoplasma, Ureaplasma, Legionella, Shigella, Salmonella, pathogenic Escherichia coli species, Bordatella, Neisseria, Treponema, Bacillus, Haemophilus, Moraxella, Vibrio, Staphylococcus spp., each of which may be resistant to one or more antibiotics.

In another embodiment, the compositions, uses and methods described herein include treating an infection by administering one or more monomer peptides and one or more other antibiotics.

Illustrative other antibiotics that may be included in the compositions, uses, and methods described herein include, but are not limited to, aminoglycosides, antimicrobial peptides, carbapenems, cephalosporins, cephems, glycoproteins, fluoroquinolones/quinolones, oxazolidinones, penicillins, streptogramins, sulfonamides, and tetracyclines.

Illustrative aminoglycosides include, but are not limited to, amikacin, gentamycin, tobramycin, netromycin, streptomycin, kanamycin, paromomycin, and neomycin.

Illustrative antimicrobial peptides include, but are not limited to, colistin, topical polymyxin B, daptomycin, gramicidin, pexiganan (magainin), omiganan, iseganan, mefloquine, venturicidin A, antimycin, myxothiazol, stigmatellin, diuron, iodoacetamide, potassium tellurite hydrate, aDL-vinylglycine, N-ethylmaleimide, L-allyglycine, diaryquinoline, betaine aldehyde chloride, acivcin, psicofuraine, buthionine sulfoximine, diaminopemelic acid, 4-phospho-D-erythronhydroxamic acid, motexafin gadolinium and xycitrin.

Illustrative carbapenems include, but are not limited to, imipenem/cilastatin sodium, meropenem, ertapenem, and panipenem/betamipron.

Illustrative cephalosporins include, but are not limited to, cefixime, cefpodoxime, ceftibuten, cefdinir, cefaclor, cefprozil, loracarbef, cefadroxil, cephalexin, and cephradineze.

Illustrative cephems include, but are not limited to, cefepime, cefpirome, cefataxidime pentahydrate, ceftazidime, ceftriaxone, ceftazidime, cefotaxime, cefteram, cefotiam, cefuroxime, cefamandole, cefuroxime axetil, cefotetan, cefazolin sodium, cefazolin, and cefalexin.

Illustrative fluoroquinolones/quinolones include, but are not limited to, ciproflaxacin, levofloxacin, and ofloxacin, gatifloxacin, norfloxacin, lomefloxacin, trovafloxacin, moxifloxacin, sparfloxacin, gemifloxacin, and pazufloxacin.

Illustrative glycopeptides include, but are not limited to, include vancomycin, teicoplanin, and daptomycin.

Illustrative oxazolidinones include, but are not limited to, linezolid.

Illustrative penicillins include, but are not limited to, penicillin, amoxicillin, amoxicillin-clavulanate, ampicillin, ticarcillin, piperacillin-tazobactam, carbenicillin, piperacillin, mezocillin, benzathin penicillin G penicillin V potassium, methicillin, nafcillin, oxacillin, cloxacillin, and dicloxacillin.

Illustrative streptogramins include, but are not limited to, quinupristin/dafopristin and pristinamycin.

Illustrative sulphonamides include, but are not limited to, co-trimoxazole, sulfamethoxazole trimethoprim, sulfadiazine, sulfadoxine, and trimethoprim.

Illustrative tetracyclines include, but are not limited to, tetracycline, demeclocycline, minocycline, and doxycycline.

Illustrative other antimicrobial agents and antibiotics contemplated herein (some of which are listed above) include, but are not limited to; abrifam; acrofloxacin; aptecin, amoxicillin plus clavulonic acid; apalcillin; apramycin; astromicin; arbekacin; aspoxicillin; azidozillin; azlocillin; aztreonam; bacitracin; benzathine penicillin; benzylpenicillin; clarithromycin, carbencillin; cefaclor; cefadroxil; cefalexin; cefamandole; cefaparin; cefatrizine; cefazolin; cefbuperazone; cefcapene; cefdinir; cefditoren; cefepime; cefetamet; cefixime; cefinetazole; cefminox; cefoperazone; ceforanide; cefotaxime; cefotetan; cefotiam; cefoxitin; cefpimizole; cefpiramide; cefpodoxime; cefprozil; cefradine; cefroxadine; cefsulodin; ceftazidime; ceftriaxone; cefuroxime; cephalexin; chloramphenicol; chlortetracycline; ciclacillin; cinoxacin; clemizole penicillin; cleocin, cleocin-T, cloxacillin; corifam; daptomycin; daptomycin; demeclocycline; desquinolone; dibekacin; dicloxacillin; dirithromycin; doxycycline; enoxacin; epicillin; ethambutol; gemifloxacin; fenampicin; finamicina; fleroxacin; flomoxef; flucloxacillin; flumequine; flurithromycin; fosfomycin; fosmidomycin; fusidic acid; gatifloxacin; gemifloxaxin; isepamicin; isoniazid; josamycin; kanamycin; kasugamycin; kitasamycin; kalrifam, latamoxef; levofloxacin, levofloxacin; lincomycin; linezolid; lomefloxacin; loracarbaf; lymecycline; mecillinam; methacycline; methicillin; metronidazole; mezlocillin; midecamycin; minocycline; miokamycin; moxifloxacin; nafcillin; nafcillin; nalidixic acid; neomycin; netilmicin; norfloxacin; novobiocin; oflaxacin; oleandomycin; oxacillin; oxolinic acid; oxytetracycline; paromycin; pazufloxacin; pefloxacin; penicillin g; penicillin v; phenethicillin; phenoxymethyl penicillin; pipemidic acid; piperacillin and tazobactam combination; piromidic acid; procaine penicillin; propicillin; pyrimethamine; rifadin; rifabutin; rifamide; rifampin; rifapentene; rifomycin; rimactane, rofact; rokitamycin; rolitetracycline; roxithromycin; rufloxacin; sitafloxacin; sparfloxacin; spectinomycin; spiramycin; sulfadiazine; sulfadoxine; sulfamethoxazole; sisomicin; streptomycin; sulfamethoxazole; sulfisoxazole; quinupristan-dalfopristan; teicoplanin; temocillin; gatifloxacin; tetracycline; tetroxoprim; telithromycin; thiamphenicol; ticarcillin; tigecycline; tobramycin; tosufloxacin; trimethoprim; trimetrexate; trovafloxacin; vancomycin; verdamicin; azithromycin; and linezolid.

In another embodiment, the antibiotic is a polymyxin, e.g., colistin. In another embodiment, the antibiotic is a carbapenem, e.g., imipenem.

Chex1-Arg20 and analogs, and derivatives thereof, may be prepared by conventional and routine processes. For example, the peptide portion may be chemically synthesized using solid phase synthesis methods. In one method, the synthesis and analysis of peptides are described in Otvos et al., J. Med. Chem. 48:5349 (2005) and Cudic et al., Peptides 23:271 (2002).

Peptide monomers may be made on a standard automated synthesizer. Peptide monomers may be detached from the resin and may be purified by reverse phase high pressure liquid chromatography. Matrix-assisted laser desorption/ionization (MALDI)-MS may be used to verify the accuracy of the sequences and their purity.

In another embodiment, the monomer peptides, such as Chex1-Arg20 or analogs and derivatives thereof, and antibiotic are administered directly to a host animal. In another embodiment, the compounds will be suspended in a pharmaceutically-acceptable carrier (e.g., physiological saline) and administered orally or by intravenous infusion, or administered subcutaneously, intramuscularly, intrathecally, intraperitoneally, intrarectally, intravaginally, intranasally, intragastrically, intratracheally, or intrapulmonarily. In another embodiment, the intratracheal or intrapulmonary delivery can be accomplished using a standard nebulizer, jet nebulizer, wire mesh nebulizer, dry powder inhaler, or metered dose inhaler. They can be delivered directly to the site of the disease or disorder, such as lungs, kidney, or intestines. The dosage required depends on the choice of the route of administration; the nature of the formulation; the nature of the patient's illness; the host animal's size, weight, surface area, age, and sex; other drugs being administered; and the judgment of the attending physician. Suitable dosages are in the range of 0.01-100.0 μg/kg. Wide variations in the needed dosage are to be expected in view of the variety of antibiotics available and the differing efficiencies of various routes of administration. For example, oral administration would be expected to require higher dosages than administration by i.v. injection. Variations in these dosage levels can be adjusted using standard empirical routines for optimization as is well understood in the art. Administrations can be single or multiple (e.g., 2-, 3-, 4-, 6-, 8-, 10-; 20-, 50-, 100-, 150-, or more fold). Encapsulation of the Chex1-Arg20 and analogs, and derivatives thereof, and antibiotic in a suitable delivery vehicle (e.g., polymeric microparticles or implantable devices) may increase the efficiency of delivery, such as for oral delivery.

In another embodiment, kits comprising monomer peptides and other antibiotics are described. The monomer peptides, such as Chex1-Arg20 or analogs and derivatives thereof, and antibiotic may be in the same container or in separate containers. The kit may further comprise additional reagents for carrying out the methods (e.g., buffers, containers, additional therapeutic agents) as well as instructions.

In another embodiment, pharmaceutical formulations and methods of administering the same to achieve any of the therapeutic effects (e.g., treatment of infection) are described. The pharmaceutical formulations may comprise one or more pharmaceutically acceptable carriers, diluents, and/or excipients, and combinations thereof.

The term “pharmaceutically acceptable” generally refers to a material that is not biologically or otherwise undesirable, i.e., the material can be administered to a host animal without causing any undesirable biological effects such as toxicity.

The formulations of the invention can optionally comprise medicinal agents, pharmaceutical agents, carriers, adjuvants, dispersing agents, diluents, and the like.

The compounds and compositions described herein can be formulated for administration in a pharmaceutical carrier in accordance with known techniques. See, e.g., Remington, The Science and Practice of Pharmacy (21^{st} Ed. 2006). In the manufacture of a pharmaceutical formulation according to the invention, the compound (including the physiologically acceptable salts thereof) is typically admixed with, inter alia, an acceptable carrier. The carrier can be a solid or a liquid, or both, and is preferably formulated with the compound as a unit-dose formulation, for example, a tablet, which can contain from 0.01 or 0.5% to 95% or 99% by weight of the compound. One or more compounds can be incorporated in the formulations of the invention, which can be prepared by any of the well-known techniques of pharmacy.

In another embodiment, methods for treating host animals in vivo are described, where the methods comprise administering to a host animal a pharmaceutical composition comprising Chex1-Arg20 or analogs and derivatives thereof, and an antibiotic in a pharmaceutically acceptable carrier, wherein the pharmaceutical composition is administered in a therapeutically effective amount. Administration of the compounds of the present invention to a human subject or an animal in need thereof can be by any means known in the art for administering compounds.

The formulations of the invention include those suitable for oral, rectal, topical, buccal (e.g., sub-lingual), vaginal, parenteral (e.g., subcutaneous, intramuscular including skeletal muscle, cardiac muscle, diaphragm muscle and smooth muscle, intradermal, intravenous, intraperitoneal), topical (i.e., both skin and mucosal surfaces, including airway surfaces), intranasal, transdermal, intraarticular, intrathecal, and inhalation administration, administration to the liver by intraportal delivery, as well as direct organ injection (e.g., into the liver, into the brain for delivery to the central nervous system, into the pancreas, or into a tumor or the tissue surrounding a tumor). The most suitable route in any given case will depend on the nature and severity of the condition being treated and on the nature of the selected compound which is being used.

For injection, the carrier will typically be a liquid, such as sterile pyrogen-free water, pyrogen-free phosphate-buffered saline solution, bacteriostatic water, or Cremophor EL[R] (BASF, Parsippany, N.J.). For other methods of administration, the carrier can be either solid or liquid.

For oral administration, the compound can be administered in solid dosage forms, such as capsules, tablets, and powders, or in liquid dosage forms, such as elixirs, syrups, and suspensions. Compounds can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that can be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for buccal (sub-lingual) administration include lozenges comprising the compound in a flavored base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the compound, which preparations are preferably isotonic with the blood of the intended recipient. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit/dose or multi-dose containers, for example sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, in one aspect of the present invention, there is provided an injectable, stable, sterile composition comprising a compound of the invention, in a unit dosage form in a sealed container. The compound or salt is provided in the form of a lyophilizate which is capable of being reconstituted with a suitable pharmaceutically acceptable carrier to form a liquid composition suitable for injection thereof into a host animal. The unit dosage form typically comprises from about 1 mg to about 10 grams of the compound or salt. When the compound or salt is substantially water-insoluble, a sufficient amount of emulsifying agent which is pharmaceutically acceptable can be employed in sufficient quantity to emulsify the compound or salt in an aqueous carrier. One such useful emulsifying agent is phosphatidyl choline.

Formulations suitable for rectal administration are preferably presented as unit dose suppositories. These can be prepared by admixing the compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which can be used include petroleum jelly, lanoline, polyethylene glycols, alcohols, transdermal enhancers, and combinations of two or more thereof.

Formulations suitable for transdermal administration can be presented as discrete patches adapted to remain in intimate contact with the epidermis of the recipient for a prolonged period of time. Formulations suitable for transdermal administration can also be delivered by iontophoresis (see, for example, Tyle, Pharm. Res. 3:318 (1986)) and typically take the form of an optionally buffered aqueous solution of the compounds. Suitable formulations comprise citrate or bis/tris buffer (pH 6) or ethanol/water and contain from 0.1 to 0.2M of the compound.

The compound can alternatively be formulated for nasal administration or otherwise administered to the lungs of a host animal by any suitable means, e.g., administered by an aerosol suspension of respirable particles comprising the compound, which the host animal inhales. The respirable particles can be liquid or solid. The term “aerosol” includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. Aerosol also includes a dry powder composition suspended in air or other carrier gas, which can be delivered by insufflation from an inhaler device, for example. See Ganderton & Jones, Drug Delivery to the Respiratory Tract, Ellis Horwood (1987); Gonda (1990) Critical Reviews in Therapeutic Drug Carrier Systems 6:273-313; and Raeburn et al., J. Pharmacol. Toxicol. Meth. 27:143 (1992). Aerosols of liquid particles comprising the compound can be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the compound can likewise be produced with any solid particulate medicament aerosol generator, by techniques known in the pharmaceutical art.

Alternatively, one can administer the compound in a local rather than systemic manner, for example, in a depot or sustained-release formulation.

Further, the present invention provides liposomal formulations of the compounds disclosed herein and salts thereof. The technology for forming liposomal suspensions is well known in the art. When the compound or salt thereof is an aqueous-soluble salt, using conventional liposome technology, the same can be incorporated into lipid vesicles. In such an instance, due to the water solubility of the compound or salt, the compound or salt will be substantially entrained within the hydrophilic center or core of the liposomes. The lipid layer employed can be of any conventional composition and can either contain cholesterol or can be cholesterol-free. When the compound or salt of interest is water-insoluble, again employing conventional liposome formation technology, the salt can be substantially entrained within the hydrophobic lipid bilayer which forms the structure of the liposome. In either instance, the liposomes which are produced can be reduced in size, such as by standard sonication and homogenization techniques.

The liposomal formulations containing the compounds disclosed herein or salts thereof, can be lyophilized to produce a lyophilizate which can be reconstituted with a pharmaceutically acceptable carrier, such as water, to regenerate a liposomal suspension.

In the case of water-insoluble compounds, a pharmaceutical composition can be prepared containing the water-insoluble compound, such as for example, in an aqueous base emulsion. In such an instance, the composition will contain a sufficient amount of pharmaceutically acceptable emulsifying agent to emulsify the desired amount of the compound. For example, useful emulsifying agents include phosphatidyl cholines and lecithin.

In another embodiment, the compound is administered to the host animal in a therapeutically effective amount, as that term is defined above. Dosages of pharmaceutically active compounds can be determined by methods known in the art, see, e.g., Remington's Pharmaceutical Sciences (Maack Publishing Co., Easton, Pa.). The therapeutically effective dosage of any specific compound will vary somewhat from compound to compound, and patient to patient, and will depend upon the condition of the patient and the route of delivery. As a general proposition, a dosage from about 0.1 to about 50 mg/kg will have therapeutic efficacy, with all weights being calculated based upon the weight of the compound, including the cases where a salt is employed. Toxicity concerns at the higher level can restrict intravenous dosages to a lower level such as up to about 10 mg/kg, with all weights being calculated based upon the weight of the compound, including the cases where a salt is employed. A dosage from about 10 mg/kg to about 50 mg/kg can be employed for oral administration. Typically, a dosage from about 0.5 mg/kg to 5 mg/kg can be employed for intramuscular injection. For example, dosages are about 1 μmol/kg to 50 μmol/kg, and more specifically to about 22 μmol/kg and to 33 μmol/kg of the compound for intravenous or oral administration, respectively.

In another embodiment, more than one administration (e.g., two, three, four, or more administrations) can be employed over a variety of time intervals (e.g., hourly, daily, weekly, monthly, etc.) to achieve therapeutic effects.

The present invention finds use in veterinary and medical applications. Suitable host animals include both avians and mammals, with mammals being preferred. The term “avian” as used herein includes, but is not limited to, chickens, ducks, geese, quail, turkeys, and pheasants. The term “mammal” as used herein includes, but is not limited to, humans, bovines, ovines, caprines, equines, felines, canines, lagomorphs, etc. Human subjects include neonates, infants, juveniles, and adults.

The invention is described further in the following examples that are intended as illustrative only since numerous modifications and variations therein will be apparent to those skilled in the art.

### EXAMPLES

Materials. Unless otherwise indicated, reagents and other materials are used as obtained from commercial suppliers. The colistin sulfate preparation (15,000 IU/mg) is obtained from Sigma-Aldrich Kft (Budapest, Hungary) and imipenem is obtained from MSD Budapest, Hungary Merck (tienamycin-formamidinemonohydrate sodium cilistatin marketed as Tienam).

### Example

Bacterial strains. The K. pneumoniae strain originated from a human wound infection at Miskolc Healthcare Center/Semmelweis University Hospital and is designated as K97/09 (Toth et al., 2010). K97/09 is a carbapenemase-expressing strain (blaKPC-2) that is extensively drug-resistant, including ceftazidime, ceftriaxone, imipenem, meropenem, ciprofloxacin, gentamicin and colistin. The A. baumannii strain (ATCC BAA-1605) originated from the tracheal aspirate of a Canadian soldier with ventilator-associated pneumonia. The strain is resistant to ceftazidime, gentamicin, piperacillin, aztreonam, cefepime, ciprofloxacin, imipenem and meropenem (Tien et al., 2007). The E. coli UNT167-1 is a carbapenem resistant strain, isolated from a chronic urinary tract infection case at the University of Texas (Zhanel et al., 2018). The B. pseudomallei strain, 1026b, was originally isolated in 1993 from a 29-year-old diabetic rice farmer in Thailand with melioidosis. MDR and highly virulent clinical isolate A. baumannii (HUMC1) was isolated from the blood and sputum of a patient at Harbor-UCLA Medical Center (Torrance, Calif.). HUMC1 is resistant to all clinically useful antibiotics reported by the clinical microbiology laboratory, except for colistin, and has been reported to be hypervirulent in experimental murine models of infection. HUMC1 is cultured using aseptic technique and routinely grown in tryptic soy broth (TSB) or on tryptic soy agar (TSA) plates.

### Example

In vitro activity and determination of MICs. Minimal inhibitory concentration (MIC) assays are performed using sterile 96-well polypropylene plates in a final volume of 100 mL. Briefly, 50 μL of midlogarithmic phase bacterial cultures is diluted to 5×10⁵ CFU/mL in Mueller-Hinton broth (MHB) and then added to 50 μL of the serially diluted antibiotic. The highest test compound concentration evaluated is illustratively 256 mg/L. Cultures are incubated at 37° C. for 16-20 h without shaking. MICs are identified as the lowest antimicrobial concentrations at which turbidity is not observed.

### Example

Fractional inhibitory concentration index (FICI) assay. Antimicrobial synergy is determined by evaluating the fractional inhibitory concentration (FICI) or (FIC) and is characterized by a conventional checkerboard assay (Fernandez-Cuenca et al., 2003) to identify combinational activity. Bacteria grown to mid-logarithmic phase in MHB is pre-incubated with serially diluted concentrations of test peptides and the antimicrobial controls, imipenem, colistin or meropenem. The FICI is determined for each combination with <0.5 indicating synergy, 0.5-4 indicating addition, and >4 indicating antagonism. The FICI assays are performed in triplicate for repeatability and to characterize assay variability. Alternatively, the sum of the FICs (ΣFIC) is calculated with the equation ΣFIC=FICA+FICB=(CA/MICA)+(CB/MICB), where MICA and MICB are the MICs of antimicrobial A and B alone, respectively, and CA and CB are the concentrations of the drugs when combined, respectively. Synergy is defined as a ΣFICs≤0.5 and additive activity is defined as a ΣFICs>0.5≤1.0.

### Example

In vivo infection models. NMRI mice weighing approximately 20 g (4 weeks old) are infected by intraperitoneal (ip) injection of 4×10⁸ CFU/g K. pneumoniae K97/09. Mice are randomly allocated to untreated (control) and treatment groups (5 mice per group).

### Example

In vivo Studies. Animals. NMRI (Naval Medical Research Institute) BR or CD-1 mice (Toxi-Coop Zrt, Budapest, Hungary) are housed in plastic type 2 cages, 3-5 mice per cage, on softwood granules as bedding. The room is kept between 21° C. and 25° C. with 12 h light:12 h dark cycles. The animals have free access to tap water and pelleted rodent food. Upon completion of the experiments, surviving mice are euthanized by diethyl ether inhalation. Animals are maintained and handled in accordance with the recommendations of the Guidelines for the Care and Use of Laboratory Animals, and the protocols are approved by the Animal Care Committee of Semmelweis University.

### Example

Bacteremia synergy Assay 1. Test animals are randomized into the following untreated (control) and treatment groups:
Group 1: phosphate buffered saline (PBS) subcutaneously (sc) 1 h after infection.
Group 2: imipenem 30 mg/kg sc at 2, 14 and 26 h after infection.
Group 3: test monomer 1 mg/kg im 1, 13 and 25 h after infection, imipenem 30 mg/kg sc at 2, 14 and 26 h after infection.
Group 4: colistin 10 mg/kg sc at 2, 14 and 26 h after infection.
Group 5: test monomer peptide 1 mg/kg im 1, 13 and 25 h after infection, colistin 10 mg/kg sc at 2, 14 and 26 h after infection.
Group 6: test monomer peptide 0.5 mg/kg im 1, 13 and 25 h after infection, colistin 10 mg/kg sc at 2, 14 and 26 h after infection.
Group 7: test monomer peptide 1 mg/kg im 1, 13 and 25 h after infection, colistin 1 mg/kg sc at 2, 14 and 26 h after infection.
Group 8: test monomer peptide 0.5 mg/kg im 1, 13 and 25 h after infection, colistin 1 mg/kg sc at 2, 14 and 26 h after infection.
Group 9: colistin 10 mg/kg sc 2 h after infection, test monomer peptide 1 mg/kg im 5 h after infection.
   Survival is recorded hourly 24-36 h after infection. Blood samples (10 μL) are taken from the tail vein to determine the bacterial burden at 6 and 30 h after infection from all surviving animals. Groups with 2 or more animal having blood bacterial counts below the level of detection (1×10³ CFU/mL) at 6 h post-infection are excluded from analysis due to presumption of low inoculum or rapid host clearance. The blood is prevented from coagulation with EDTA and the samples are serially diluted in 0.9% saline. Each dilution is cultured providing a detectable threshold of 10³ CFU/mL.

### Example

Bacteremia synergy Assay 2. Test animals are randomized into the following untreated (control) and treatment groups:
Group 1: PBS sc 1 h after infection.
Group 2: Colistin 1 mg/kg sc at 2 and 13 h after infection.
Group 3: test monomer peptide 1 mg/kg im 1 and 12 h after infection.
Group 4: test monomer peptide 1 mg/kg im 1 and 12 h after infection, colistin 1 mg/kg sc at 2 and 13 h after infection.
Group 5: Colistin 10 mg/kg sc 4 h after infection, A3-APO 1 mg/kg im 6 h after infection.
Group 6: test monomer peptide 0.5 mg/kg im 1 and 12 h after infection.
Group 7: test monomer peptide 0.5 mg/kg ip 1 and 12 h after infection.
Group 8: test monomer peptide 0.5 mg/kg im 1 and 12 h after infection, colistin 1 mg/kg sc at 2 and 13 h after infection.
   Survival is monitored at 12, 24 and 36 h and blood samples are taken 4 and 11 h after infection and worked up as in Assay 1. Blood bacterial load reduction and survival in the various groups are compared with Chi-square and unpaired Student's t-testing, respectively (Microsoft Excel, Microsoft, 2007, Redmond, Wash., USA, and SlideWrite, Encinitas, Calif., USA).

### Example

Bacteremia synergy assay 3. Test animals are infected iv at the ID₉₅ (10⁸ CFU/mouse) of A. baumannii HUMC1 strain as independently determined. 24 hr post-infection, animals are randomized into groups (6-8 animals/group) to receive one of the following regimens: i) untreated controls; ii) imipenem/cilastatin (IPM/CIL) alone at 40 mg/kg, im, bid; iii-v) test monomer peptide, such as Chex1-Arg20 (ARV-1502) at 1.25, 2.5 or 5.0 mg/kg, iv, bid; or vi-viii) test monomer peptide, such as Chex1-Arg20 at 1.25, 2.5 or 5.0 mg/kg, iv, bid in combination with IPM/CIL at 40 mg/kg, im, bid. Test animals are treated for three days. 24 hr after the last administration, animals are sacrificed and target tissues (e.g., kidney, spleen, liver, and blood) are removed and quantitatively cultured. Bacterial counts in the target tissues are calculated from each group and expressed as mean (±SD) log₁₀ CFU/g. tissues or log₁₀ CFU/ml of blood.

### Example

In vitro synergistic activity. Checkerboard assays are conducted to identify synergistic outcomes with illustrative antibiotics in combination with monomer peptides, as shown in the following table.

| | | ARV1502 + | ARV1502 + | ARV1502 + | ARV1502 + |
|---|---|---|---|---|---|
| Organism | Strain | Imipenem | Colistin | Tigecycline | Piperacillin |
| A. baumanii | ATCC 17961 | ADD | SYN | ADD | SYN |
| A. baumanii | UCLA strain | ADD | SYN | SYN | SYN |
| (XDR) | HUM-C | | | | |
| E. Coli | BAA-2469 | ADD | SYN | ADD | SYN |
| NDM-1(+) | | | | | |
| E coli | ATCC-43895 | NT | SYN | SYN | SYN |
| 0157:H7 | | | | | |
| P. aeruginosa | ATCC 15692 | ADD | SYN | ADD | SYN |
| P. aeruginosa | ATCC BAA-47 | SYN | SYN | ANT | — |
| K. pneumoniae | BAA-2146 | ADD | SYN | ADD | — |
| NDM-1(+) | | | | | |
| K. pneumoniae | BAA-1905 | ADD | ADD | ADD | — |
| KPC (+) | | | | | |

ΣFIC < 0.5 = synergy (SYN); ΣFIC 0.5-4.0 = additivity (ADD); ΣFIC > 4.0 = antagonism (ANT); NT = not tested.

| | | MIC of | MIC of | |
|---|---|---|---|---|
| | | antibiotic | antibiotic + | |
| | Antibiotic | alone | ARV-1501 | ΣFIC |
| K. pneumoniae | colistin | 64 | 1 | 0.08 |
| K97/09 | imipenem | >256 | 4 | 0.5 |

### Example

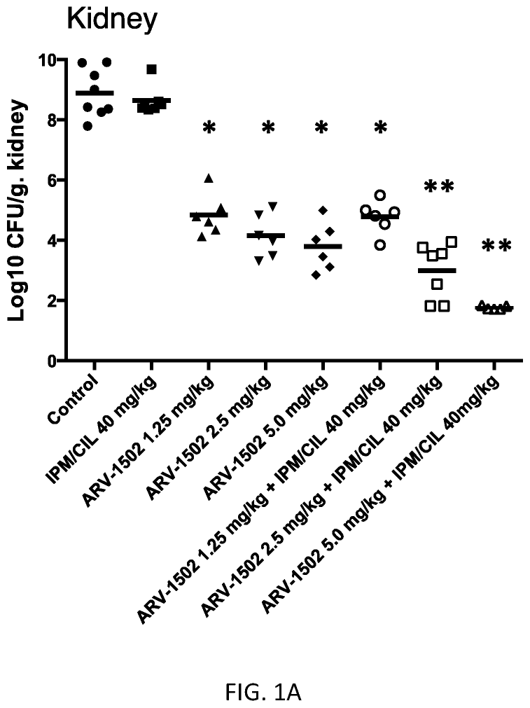

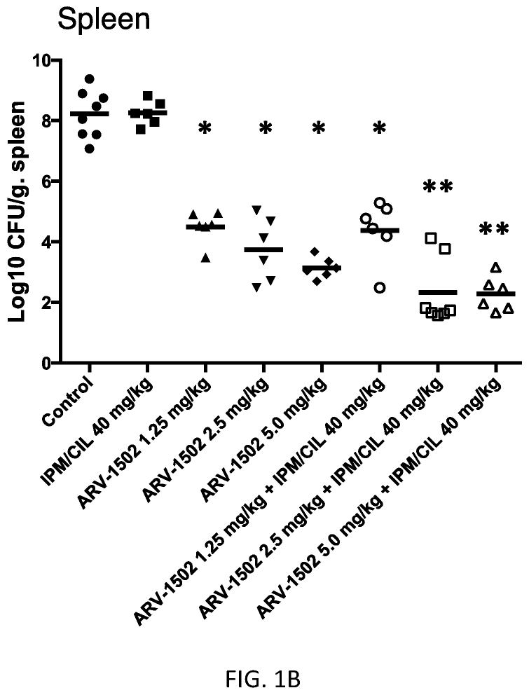

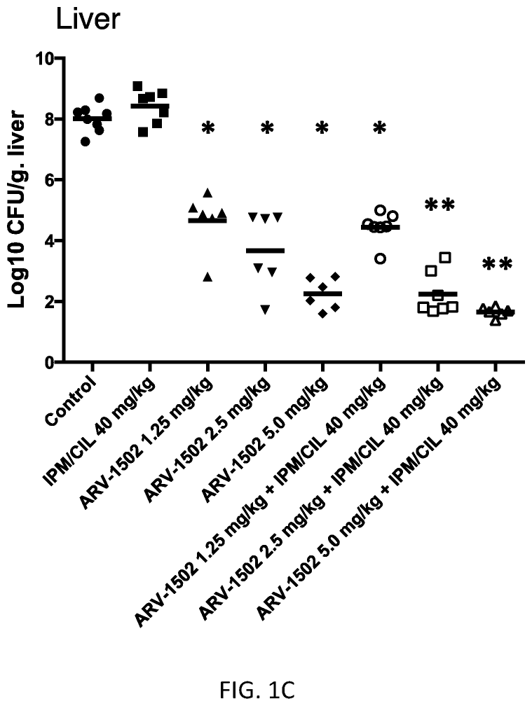

ARV-1502 evaluation using bacteremia synergy assay 3. All ARV-1502+IPM/CIL treatment groups showed significantly reduced A. baumannii densities in the target tissues in a dose-dependent manner vs. untreated control and IPM/CIL alone treated groups, as shown in FIGS. 1A (kidney), 1B (spleen), and 1C (liver). IPM/CIL alone failed to impact bacterial density in any of the target tissues as compared to the control group.

### Example

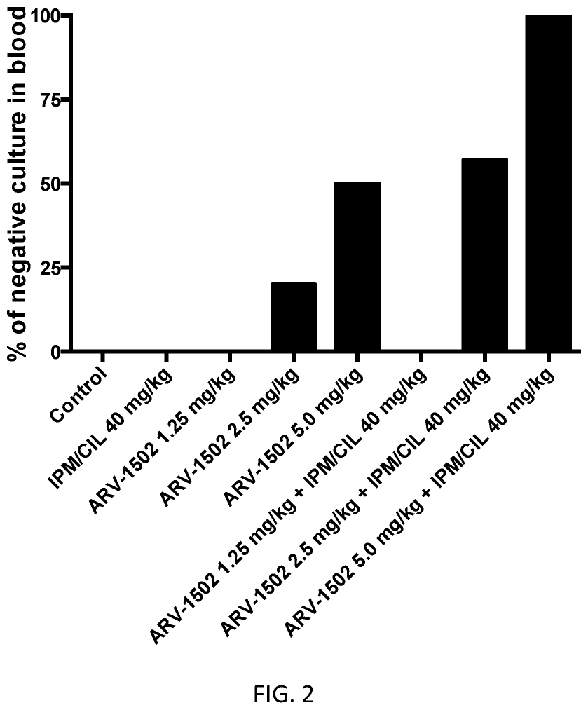

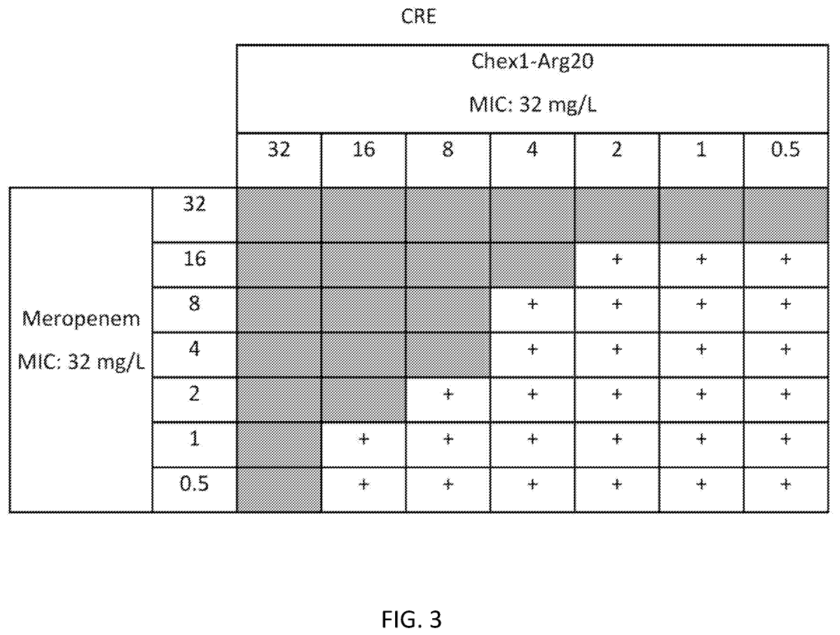

ARV-1502 showed dose-dependent negative blood culture of A. baumannii. Except for the lowest dose, each ARV-1502 treatment group also showed a complete response (negative blood culture) in a portion of the test animals, and the ARV-1502 5.0 mg/kg treatment group showed a complete response in all (100%) test animals, as shown in FIG. 2.

### Example

In vitro activity and synergy. MIC values of monomer peptides described herein, colistin, and imipenem against the K. pneumoniae strain (K97/09) were typically ˜32 mg/L, and 64 mg/L, and >256 mg/L, respectively. MIC values of colistin and imipenem against the A. baumannii strain (BAA-1605) were <0.5 mg/L and 64 mg/L, respectively. Combining antimicrobials against K97/09 results in synergy. Combining imipenem and monomer peptides described herein against BAA-1605 (colistin is not evaluated because BAA-1506 is a colistin-sensitive strain) results in synergy. The negative control Allo-aca peptide or peptide Gly11, an A3-APO analog that fails to bind bacterial DnaK (Cassone et al., 2008), had no activity on either pathogen (MICs>256 mg/L), and failed to exert any improvement in the MIC values when added together with either imipenem or colistin suggesting that the effect is specific and can be correlated with DnaK inhibition resulting in inhibition of resistance enzymes.

### Example

Synergy evaluation in E. coli. The MIC of both meropenem and Chex1-Arg20 against the E. coli UNT167-1 strain was determined to be 32 mg/L. When added together, the peptide and the carbapenem showed synergism (ΣFIC=0.38, FIG. 2) and efficacy at doses that are considered to be subtherapeutic for the corresponding monotherapy.

### Example

Post-antibiotic effects. Chex1-Arg20 exhibits extended post-antibiotic effects in vivo. Mice are infected with an extended spectrum β-lactamase expressing E. coli strain ip and treated with 2.5, 5 and 10 mg/kg of Chex1-Arg20, administered ip. During the 12-h treatment period a dose of 2.5 mg/kg Chex1-Arg20 reduced the blood bacterial load by 1.5-2 log 10 units; by 24 h the improvement was not statistically significant, as shown I the following table.

Efficacy of peptide Chex1-Arg20 treatment in mice challenged intraperitoneally (ip) with E. coli 5770 as represented by reduction of blood bacterial counts.

| Treatment | Bacterial counts in blood (CFU/mL) after | | | | |
|---|---|---|---|---|---|
| with Chex1- | inoculation/challenge | | | | |
| Arg20 | 4 h* | 8 h* | 12 h* | 16 h | 24 h |
| Untreated | 3.68 × 10⁷ | 6.55 × 10⁷ | 1 × 10⁸ | 2.4 × 10⁷ | <3 × 10⁵ |
| 2.5 mg/kg | 3.68 × 10⁷ | 4.2 × 10⁶ | <1.7 × 10⁶ | <1 × 10⁶ | <4.8 × 10⁵ |
| | | (0/3) | (1/3) | (1/3) | (1/3) |
| 5 mg/kg | 3.68 × 10⁷ | 4.3 × 10⁵ | 1.1 × 10⁶ | <1.1 × 10⁵ | <1 × 10³ |
| | | (0/3) | (0/3) | (2/3) | (3/3) |
| 10 mg/kg | 3.68 × 10⁷ | 2.4 × 10⁵ | 2.9 × 10⁵ | <1 × 10³ | <1 × 10³ |
| | | (0.3) | (0.3) | (3/3) | (3/3) |

The peptide was administered ip 4, 8 and 12 h after challenge (*). Blood was taken immediately before antimicrobial treatments. Bacterial counts were determined from 3 mice in each group. The numbers in parentheses represent the number of animals with bacterial counts below the detection limit of 1,000 CFU/mL.

However, at 5 mg/kg Chex1-Arg20 treatment demonstrated a >2 log 10 CFU/mL reduction after inoculation and complete sterilization of the blood at 24 h. At the highest dose of 10 mg/kg, the blood was sterilized by 4 h after the last peptide treatment.

### Example

Melioidosis model. Mean inhaled doses of 58×LD50 (2 separate sprays of 56 and 60 LDs) of Burkholderia pseudomallei 1026b are administered to 6-8 week-old female Balb/c mice by whole-body aerosol. Aerosol is generated using a three-jet collision nebulizer. All aerosol procedures are controlled and monitored using the Automated Bioaerosol Exposure system (Hartings and Roy, 2004) operating with a whole-body rodent exposure chamber. Integrated air samples are obtained from the chamber during each exposure using an all-glass impinger. Mice are randomly placed into separate cages upon the conclusion of each aerosol. Cohort size for statistical evaluation is 10 mice. Ceftazidime is administered ip at 300 (Group 1) or 150 mg/kg (Group 2) doses beginning 24 h post-challenge four times a day and treatment continued for 21 days. Three additional groups receiving 150 mg/kg ceftazidime ip are treated simultaneously with 2.5, 5 or 10 mg/kg test monomer peptide, such as Chex1-Arg20, or an analog or derivative thereof, added im or iv (Groups 3-5) co-administered with ceftazidime ip. A vehicle control group receives 0.2 mL saline sc four times a day. Survival is monitored twice daily during treatment and once daily thereafter. Moribund animals are euthanized as necessary and counted as dead. In accordance with the protocol approved by the Institutional Animal Care and Use Committee of the United States Army Medical Research Institute of Infectious Diseases, the study is terminated at day 62. At the conclusion of the study all animals are humanely euthanized and target organs (spleens and lungs) are harvested for the determination of bacterial loads. The results are processed with a stratified Kaplan-Meyer analysis with a logᵣₐₙₖ test as implemented on Prism Version 5.04 GraphPad.

### Example

Long-term synergy in a melioidosis model. Melioidosis is a Centers for Disease Control and Prevention (CDC) Category B bioterrorism disease and requires prolonged treatment with a high failure rate. In the murine model untreated mice died after 4 days. Chex1-Arg20 monotherapy failed to rescue any mouse, as shown in the following table.

Treatment success in mice infected with Burkholderia pseudomallei 1026b.

| | | | | B. pseudomallei load in |
|---|---|---|---|---|
| | | | | survivals (CFU/spleen)- |
| | Dose (mg/kg)- | Number | | (total number of samples |
| | administration | of | Median survival (days | analyzed/samples with |
| Antibiotic | route | deaths | post-challenge) | CFU > 10⁸) |
| Untreated | Saline-sc | 10 | 4 | No survival |
| Ceftazidime | 300-ip | 3 | (>50% survival) | 2.4 × 10⁵-(3/1) |
| Ceftazidime | 150-ip | 6 | 56.5 | 2.6 × 10⁵-(4/1) |
| Chex1-Arg20 | 5-im | 10 | 4 | No survival |
| Ceftazidime + | 150 ip + 2.5 im | 4 | (>50% survival) | 3.1 × 10⁶-(6/1) |
| Chex1-Arg20 | | | | |
| Ceftazidime + | 150 ip + 5 im | 6 | 56.5 | 8.6 × 10⁶-(4/0) |
| Chex1-Arg20 | | | | |
| Ceftazidime + | 150 ip + 10 im | 7 | 50 | 7.6 × 10⁶-(3/0) |
| Chex1-Arg20 | | | | |

The antibiotics are administered for 21 days, every 6 h, beginning 24 h post-challenge. After completion of the assay at day 62, surviving animals were euthanized and their spleens removed for bacterial count determination. The values in the last column are the mean of samples with <10⁸ CFU/spleen.

Use of a 300 mg/kg therapeutic dose (allometrically scaled from 30 mg/kg human dose) of ceftazidime monotherapy resulted in a 70% survival rate at the 62-day endpoint. When the dose of ceftazidime was reduced to 150 mg/kg, survival declined to 40%. It was found that co-administration of a lower ceftazidime dose of 150 mg/kg with 2.5 mg/kg Chex1-Arg20 increased the survival rate to 60%. Higher combination peptide doses (5 or 10 mg/kg) did not appear to provide greater survival than the 150 mg ceftazidime monotherapy.

In the spleen bacterial counts of surviving animals in the three groups that received Chex1-Arg20 combinations, only the 2.5 mg/kg group had a mouse with >10⁸ CFU raising the mean bacterial load in this (in survival terms) successful group above those in the two other (survival terms) unsuccessful treatment groups. Removing this single outlier from the analysis (six total samples in the group) the 2.5 mg/kg peptide combination group performed best in terms of spleen bacterial load. The spleen bacterial counts in the peptide combination treatment groups did not appear to be lower than those in the 150 mg/kg ceftazidime monotherapy group. It was found that the mortality benefit may not be accompanied by a bacterial load reduction in surviving animals.

### Example

Restoring antibiotic sensitivity to resistant bacteria. A colistin-resistant, carbapenem-resistant strain of K. pneumonia (K97/09) from a clinical isolate is used to test the effect of Chex1-Arg20, and analogs and derivatives thereof, treatment on antibiotic resistance.

### Example

The combination of Chex1-Arg20, and analogs and derivatives thereof, with other antibiotics is tested in a mouse model of systemic Klebsiella pneumoniae infection. The results are based on survival and blood bacterial counts.

### Example

Post-antibiotic Effect (PAE). ARV-1502 shows a surprisingly high PAE of 14 hours, which is also specifically surprising compared to quinolone antibiotics (1 hour).

### Example

Cytotoxicity of monomer peptides. In vitro cytotoxicity is determined via the Promega CellTiter 96 Aqueous Non-Radioactive Cell Proliferation Assay¹⁴ using the mammalian cell lines, HEK-293 (ATCC CRL 1573) and H-4-II-E (ATCC CRL-1548). The proliferation of HEK-293 (ATCC® CRL-1573™) and H-4-II-E (ATCC® CRL-1548™) cells is tested with monomer peptides measured at 490 nm. Cytotoxicity against HEK-293 and H-4-II-E cells was not observed for any of Examples 1-8 at 100 μM, except Example 6, which showed low H-4-II-E cytotoxicity at 48.1±3.4 μM.

### Example

Immunogenicity. ARV-1502 is inoculated into mice subcutaneously at 5 mg/kg twice in a 3-wk interval, and 4 weeks after the second immunization the blood is analyzed for antibody production. Neither IgG1, IgG2a, IgG3, IgM, nor IgE are observed by ELISA (ELISA readings between 0.09 and 0.13 AUFS units). All positive controls except IgE showed absorbance values over 0.23 AUFS (0.23-0.62).

### Comparative Example

Antibacterial activity of peptide monomers. Antibacterial assays are undertaken to determine the minimal inhibitory concentrations (MIC). Briefly, a panel of Gram-negative nosocomial bacteria, E. coli ATCC 29222, K. pneumoniae ATCC13883, A. baumannii ATCC 19606, MDR A. baumannii HUMC1, and P. aeruginosa ATCC 47085 is chosen to test the antibacterial activities of the monomers, analogs, and derivatives described herein, with 2.5×10⁵ cells/ml in MHB at 37° C. immediately prior to the determination of MIC, as shown in the following table.

Antibacterial activity, MIC (μM), against Gram-negative nosocomial pathogens

| | E. Coli | K. Pneumoniae | A. Baumannii | P. aeruginosa |
|---|---|---|---|---|
| Example | ATCC 29222 | ATCC 13883 | ATCC 19606 | ATCC 47085 |
| 1 | 2.6 ± 0.7 | 0.8 ± 0.0 | >100 | 56.3 ± 4.5 |
| (Chex1- | | | | |
| Arg20) | | | | |
| 2 | 3.6 ± 0.1 | 2.0 ± 0.2 | >100 | >100 |
| 3 | 6.5 ± 2.6 | 2.2 ± 0.4 | >100 | 96.3 ± 3.2 |
| 4 | 5.6 ± 1.8 | 2.1 ± 0.0 | >100 | 82.0 ± 5.0 |
| 5 | 5.8 ± 2.2 | 1.5 ± 0.4 | >100 | 64.7 ± 2.1 |
| 6 | 6.7 ± 1.3 | 2.0 ± 0.1 | >100 | >100 |
| 7 | 4.0 ± 1.5 | 1.7 ± 0.1 | 52.5 ± 9.5 | 28.9 ± 2.7 |
| 8 | 1.6 ± 0.1 | 1.2 ± 0.3 | 68.3 ± 1.9 | 35.3 ± 5.2 |

### Comparative Example

Co-administration of dimer peptides. An in vitro assay measuring visible bacterial growth in the presence of peptide dimers and imipenem or colistin is performed. The results for comparative peptide dimer A3-APO are shown in the following table.

Activity of combinations of dimer peptides and imipenem or colistin against multidrug-resistant bacterial strains Klebsiella pneumoniae K97/09 (KPC), Acinetobacter baumannii BAA-1805 (MACI), and Acinetobacter baumannii BAA-1605.

| | KPC | | MACI | |
|---|---|---|---|---|
| | MIC | | MIC | |
| Antibiotic | (mg/L) | KPC FIC | (mg/L) | MACI FIC |
| A3-APO | 32-64 | | 32-64 | |
| Colistin | 64 | | <0.5 | |
| A3-APO + | | 0.035 | | No growth up to |
| colistin | | Synergy | | 0.5 mg/L |
| Imipenem | >256 | | 64 | |
| A3-APO + | | 0.531 | | No growth up to |
| imipenem | | Additive | | 0.5 mg/L |
| Gly11 | >256 | | >256 | |
| Gly11 + | | 2 | | No growth up to |
| colistin | | No improvement | | 0.5 mg/L |
| | | over colistin | | |
| Gly11 + | | N/A | | 2 |
| imipenem | | No observed | | |
| | | growth inhibition | | |

Additional comparative data is disclosed in PCT international application No. PCT/US2018/66002.

### Compound Examples

Materials. 9-Fluoroenylmethoxylcarbonyl (Fmoc)-L-amino acids, 2-(6-chloro-1H-benzotriazole-1-yl)-1,1,3,3-tetramethylamonium hexafluorophosphate (HCTU), and 2-chlorotrityl chloride resin are purchased from GL Biochem (Shanghai, China). TentaGel-MB-RAM-resin is from Rapp Polymere (Tubingen, Germany). N,N-Diisopropylethylamine (DIPEA), dimethylformamide (DMF), and trifluoroacetic acid (TFA) are obtained from Auspep (Melbourne, Australia). 1,6-Bimaleimidohexane is obtained from TCL (Gillman, Australia). Isobutyl chloroformate (IBCF), NaBH₄, ethyl acetate, 2,3-dibromomaleimide, α,α′-dibromo-p-xylene, trans-1,4-dibromo-2-butene, piperidine, triisopropylsilane (TIPS), anisole, 3,6-dioxa-1,8-octanedithiol (DODT), hydrazine monohydrate, and acetonitrile (CH₃CN) are all obtained from Sigma (Sydney, Australia).

### Example

Peptides.
Chex1-Arg20 [H-Chex-Arg-Pro-Asp-Lys-Pro-Arg-Pro-Tyr-Leu-Pro-Arg-Pro-Arg-Pro-Pro-Arg-Pro-Val-Arg-NH₂] (ARV-1502)
A3-APO [(H-Chex-Arg-Pro-Asp-Lys-Pro-Arg-Pro-Tyr-Leu-Pro-Arg-Pro-Arg-Pro-Pro-Arg-Pro-Val-Arg)₂-Dab]
Gly 11 [(H-Chex-Arg-Pro-Asp-Lys-Pro-Arg-Pro-Tyr-Leu-Gly-Arg-Pro-Arg-Pro-Pro-Arg-Pro-Val-Arg)₂-Dab-NH₂]
Allo-aca, negative control leptin receptor antagonist (Otvos et al., 2011)
   Gly11, which has the same amino acid sequence as A3-APO except for a change in one residue, fails to bind DnaK and was used to validate DnaK binding as critical to the mechanism of action of A3-APO (Cassone et al., 2008). “Dab” is divalent 2,4-diamino-butyric acid. Additional details are described in U.S. provisional application Nos. 62/599,828 and 62/697,001.

### Example

Peptide preparation. The peptides are synthesized by Fmoc/tBu solid-phase methods²⁴. Dimeric peptide synthesis is carried out on a CEM Liberty microwave-assisted synthesizer using TentaGel-MB-RAM-resin or 2-chlorotrityl chloride resin as previously described¹⁴ (Scheme 1). The C-terminal modified peptides are prepared on 2-chlorotrityl chloride resin functionalized with hydrazide or N-Fmoc-amino acid alcohol prior to SPPS. Standard Fmoc-chemistry is used throughout with a 4-fold molar excess of the Fmoc-protected amino acids in the presence of 4-fold HCTU and 8-fold DIPEA. The peptides are cleaved from the solid support resin with TFA in the presence of anisole, TIPS and DODT as scavenger (ratio 95:2:2:1) for 2 h at room temperature. After filtration to remove the resin, the filtrate is concentrated under a stream of nitrogen, and the peptide products are precipitated in ice-cold diethyl ether and washed three times. The peptides are then purified by reversed-phase high performance liquid chromatography (RP-HPLC) in water and acetonitrile containing 0.1% TFA. The final products are characterized by both RP-HPLC and matrix-assisted laser desorption/ionization time-of-flight mass spectrometry (MALDI-TOF MS).

### Example

Photoluminescence. Spectra are obtained using a HORIBA Jobin Yvon Fluorolog fluorescence spectrophotometer. To minimise reabsorption effects, the optical absorptions of the sample solutions are kept around 0.10 at the excitation wavelength. The sample solutions of peptide monomer are prepared in MilliQ water in BRAND® disposable cuvettes and spectra are recorded immediately after the UV-vis absorbance measurements.

### Example

Diffusion NMR spectroscopy. Translational diffusion coefficients of peptide monomers are measured at 298 K on a Bruker Avance II 800 MHz spectrometer using a TXI cryoprobe equipped with a single gradient (Gz). Diffusion measurements are carried out using a standard BPP-STE sequence without modification (stebpgp1s, Bruker pulse sequence library). The field gradient strength of Gz is calibrated by measuring the self-diffusion coefficient of residual H₂O in a 100% ²H₂O sample at 298.13 K²⁵ using a diffusion coefficient of 1.9×10⁻⁹ m² s⁻¹ for the residual H₂O²⁶. Spectra are processed in TOPSPIN (Version 3.2, Bruker). Diffusion coefficient, D, is determined by fitting diffusion weighted intensities of well resolved and intense peaks using the T₁/T₂ relaxation module in TOPSPIN (Version 3.2, Bruker) and SigmaPlot (version 12.5, Systat Software) to the following equation:

I=I₀ exp{−γ²s²g²δ²(Δ−δ/3−τ/2)D} (1)

where γ is the gyromagnetic ratio of protons and s, g, δ, and Δ are the shape factor, amplitude, duration and separation, respectively, of the single pair of gradient pulses, and σ being the time interval within the bipolar pulse pair. Sinusoidal shaped gradient pulses are used in the present study. The effective hydrodynamic radius of PrAMPs is subsequently estimated from experimentally measured translational diffusion coefficients using the following relationship²⁷:

Rₕ=Rₕ^{REF} (D^{REF}/D) (2)

where Rₕ^{REF} and D^{REF} are the hydrodynamic radius and translational diffusion coefficient, respectively, of the reference molecule. Dioxane with Rₕ^{REF} of 2.12 Å is illustratively used as the reference molecule²⁸.

### Example

Diffusion NMR. To further delineate the potential relationship between the size/shape of these peptides with different chemical tethers in isotropic solution and their activity against pathogens, translational diffusion coefficients of monomer peptides is measured using PFG-NMR. Based on the well-known Stokes-Einstein equation, Dₜ=k_{B}T/(6πηRₕ), the molecules with larger hydrodynamic radius Rₕ are expected to display a slower diffusion coefficient D in solution.

### Example

Circular dichroism (CD) spectroscopy. As an indication of the secondary structures of peptide monomers described herein, circular dichroism (CD) spectroscopy is used.

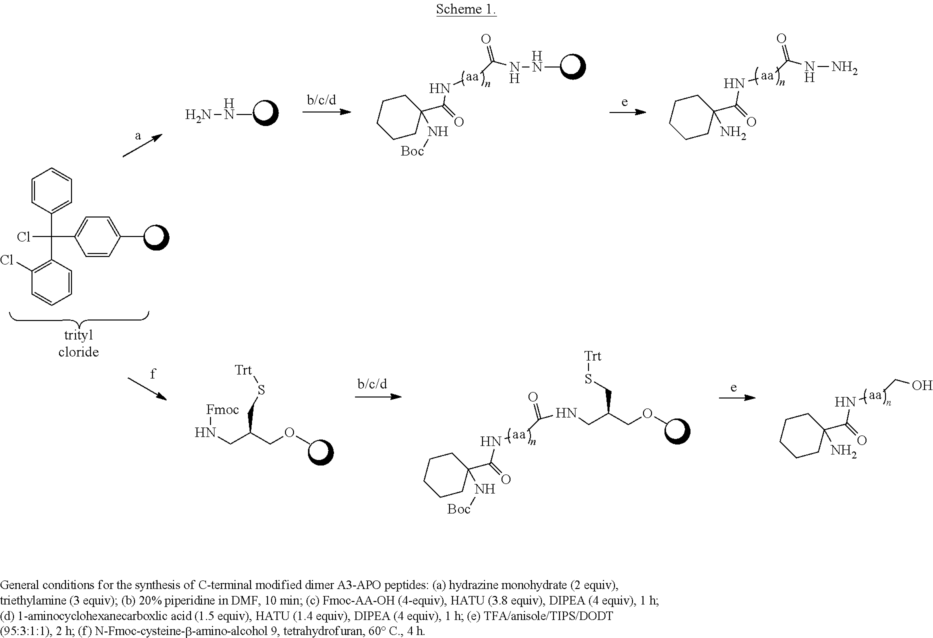

The foregoing is illustrative of the invention described herein, and is not to be construed as limiting thereof. The invention may also be embodied in different forms and should not be construed as limited to the embodiments set forth herein. The invention is also defined by included drawing, and the following claims, with equivalents of the claims to be included therein.

The following publications, and each of the additional publications cited herein are incorporated herein by reference:
1. World Health Organization, 2014.
2. A. L. Hilchie, K. Wuerth and R. E. W. Hancock, Nat. Chem. Biol., 2013, 9, 761-768.
3. L. Otvos, Jr., J. D. Wade, F. Lin, B. A. Condie, J. Hanrieder and R. Hoffmann, J. Med. Chem., 2005, 48, 5349-5359.
4. P. Czihal, D. Knappe, S. Fritsche, M. Zahn, N. Berthold, S. Piantavigna, U. Muller, S. Van Dorpe, N. Herth, A. Binas, G. Kohler, B. De Spiegeleer, L. L. Martin, O. Nolte, N. Strater, G. Alber and R. Hoffmann, ACS Chem. Biol., 2012, 7, 1281-1291.
5. F. Guida, M. Benincasa, S. Zahariev, M. Scocchi, F. Berti, R. Gennaro and A. Tossi, J. Med. Chem., 2015, 58, 1195-1204.
6. W. Li, J. Tailhades, N. O'Brien-Simpson, F. Separovic, L. Otvos, Jr., M. A. Hossain and J. Wade, Amino Acids, 2014, 46, 2287-2294.
7. G. Kragol, S. Lovas, G. Varadi, B. A. Condie, R. Hoffmann and L. Otvos, Jr., Biochemistry, 2001, 40, 3016-3026.
8. M. Scocchi, C. Lüthy, P. Decarli, G. Mignogna, P. Christen and R. Gennaro, Int. J. Pept. Res. Ther., 2009, 15, 147-155.
9. A. Krizsan, D. Volke, S. Weinert, N. Strater, D. Knappe and R. Hoffmann, Angew Chem Int Ed, 2014, 53, 12236-12239.
10. A. C. Seefeldt, F. Nguyen, S. Antunes, N. Perebaskine, M. Graf, S. Arenz, K. K. Inampudi, C. Douat, G. Guichard, D. N. Wilson and C. A. Innis, Nat. Struct. Mol. Biol., 2015, 22, 470-475.
11. R. N. Roy, I. B. Lomakin, M. G. Gagnon and T. A. Steitz, Nat. Struct. Mol. Biol., 2015, 22, 466-469.
12. A. C. Seefeldt, M. Graf, N. Perebaskine, F. Nguyen, S. Arenz, M. Mardirossian, M. Scocchi, D. N. Wilson and C. A. Innis, Nucleic Acids Res., 2016, DOI: 10.1093/nar/gkv1545.
13. E. Ostorhazi, M. C. Holub, F. Rozgonyi, F. Harmos, M. Cassone, J. D. Wade and L. Otvos, Jr., Int. J. Antimicrob. Agents, 2011, 37, 480-484.
14. W. Li, N. O'Brien-Simpson, J. Tailhades, N. Pantarat, R. Dawson, L. Otvos, Jr., E. Reynolds, F. Separovic, M. Hossain and J. Wade, Chem. Biol., 2015, 22, 1250-1258.
15. W. Li, M.-A. Sani, E. Jamasbi, L. Otvos Jr, M. A. Hossain, J. D. Wade and F. Separovic, Biochim. Biophys. Acta, Biomembr., 2016, 1858, 1236-1243.
16. W. Li, J. Tailhades, M. A. Hossain, N. M. O'Brien-Simpson, E. C. Reynolds, L. Otvos, F. Separovic and J. D. Wade, Aust. J. Chem., 2015, 68, 1373-1378.
17. L.-T. T. Nguyen, M. T. Gokmen and F. E. Du Prez, Polym. Chem., 2013, 4, 5527-5536.
18. J.-K. Y. Tan and J. G. Schellinger, Therapeutic Delivery, 2015, DOI: 10.4155/tde.15.52.
19. L. M. Tedaldi, M. E. B. Smith, R. I. Nathani and J. R. Baker, Chem. Commun., 2009, DOI: 10.1039/B915136B, 6583-6585.
20. M. E. B. Smith, F. F. Schumacher, C. P. Ryan, L. M. Tedaldi, D. Papaioannou, G. Waksman, S. Caddick and J. R. Baker, J. Am. Chem. Soc., 2010, 132, 1960-1965.
21. M. P. Robin, P. Wilson, A. B. Mabire, J. K. Kiviaho, J. E. Raymond, D. M. Haddleton and R. K. O'Reilly, J. Am. Chem. Soc., 2013, 135, 2875-2878.
22. J. Collins, J. Tanaka, P. Wilson, K. Kempe, T. P. Davis, M. P. McIntosh, M. R. Whittaker and D. M. Haddleton, Bioconjugate Chem., 2015, 26, 633-638.
23. E. Jamasbi, G. D. Ciccotosto, J. Tailhades, R. M. Robins-Browne, C. L. Ugalde, R. A. Sharples, N. Patil, J. D. Wade, M. A. Hossain and F. Separovic, Biochim. Biophys. Acta, Biomembr., 2015, 1848, 2031-2039.
24. G. B. Fields and R. L. Noble, Int. J. Pept. Protein Res., 1990, 35, 161-214.
25. S. Yao, D. K. Weber, F. Separovic and D. W. Keizer, Eur. Biophys. J., 2014, 43, 331-339.
26. P. T. Callaghan, M. A. Le Gros and D. N. Pinder, J. Chem. Phys., 1983, 79, 6372-6381.
27. S. Yao, G. J. Howlett and R. S. Norton, J. Biomol. NMR, 2000, 16, 109-119.
28. D. K. Wilkins, S. B. Grimshaw, V. Receveur, C. M. Dobson, J. A. Jones and L. J. Smith, Biochemistry, 1999, 38, 16424-16431.
29. M. Mohammed, A. Mohammed, M. Mirza and A. Ghori, Int. Res. J. Pharm, 2014, 5, 7-12.
30. G. M. Eldridge and G. A. Weiss, Bioconjugate Chem., 2011, 22, 2143-2153.
31. J. Youziel, A. R. Akhbar, Q. Aziz, M. E. B. Smith, S. Caddick, A. Tinker and J. R. Baker, Org. Biomol. Chem., 2014, 12, 557-560.
32. E. Glattard, E. S. Salnikov, C. Aisenbrey and B. Bechinger, Biophys. Chem., 2016, 210, 35-44.

1. A method for treating a bacterial infection in a host animal, the method comprising administering (a) one or more monomer peptides and (b) one or more antibiotics to the host animal.

2. The method of claim 1 where the infection is a bloodstream infection.

3. The method of claim 1 where the infection is a urinary tract infection.

4. The method of claim 1 where the infection is pneumonia.

5. The method of claim 1 where the infection is caused at least in part by Gram-negative bacteria.

6. The method of claim 1 where the infection is caused at least in part by Acinetobacter.

7. The method of claim 1 where the infection is caused at least in part by Enterobacteriaceae.

8. The method of claim 1 where the infection is caused at least in part by Escherichia coli.

9. The method of claim 1 where the infection is caused at least in part by Klebsiella.

10. The method of claim 1 where the infection is caused at least in part by Legionella.

11. The method of claim 1 where the infection is caused at least in part by Pseudomonas.

12. The method of claim 1 where the infection is caused at least in part by one or more pathogenic organisms that are resistant to one or more antibiotics.

13. The method of claim 1 where the monomer peptide is a DnaK inhibitor.

14. The method of claim 1 where the monomer peptide is a host defense peptide (HDP).

15. The method of claim 1 where the one or more monomer peptides are administered with one or more beta-lactam antibiotics.

16. The method of claim 1 where the one or more monomer peptides are administered with one or more tetracyclines.

17. The method of claim 1 where the one or more monomer peptides are administered with one or more polymyxins.

18. The method of claim 1 where the one or more monomer peptides are administered with one or more aminoglycoside antibiotics.

19. The method of claim 1 where the one or more monomer peptides are administered with one or more antipseudomonal beta lactam antibiotics.


SEQUENCE LISTING (26 sequences)

SEQ ID NO: 1 - protein, 20 aa, Artificial Sequence
  Peptide
  MUTAGEN (1)..(1): 1-amino-cyclohexyl carboxyl
  AMIDATION (20)..(20): Arginine-amide
XRPDKPRPYLPRPRPPRPVX

SEQ ID NO: 2 - protein, 19 aa, Artificial Sequence
  Peptide
  ACETYLATION (1)..(1): Acetyl-Arginine
  AMIDATION (19)..(19): Arginine-amide
XPDKPRPYLPRPRPPRPVX

SEQ ID NO: 3 - protein, 19 aa, Artificial Sequence
  Peptide
  MUTAGEN (1)..(1): Butyryl-Arginine
  AMIDATION (19)..(19): Arginine-amide
XPDKPRPYLPRPRPPRPVX

SEQ ID NO: 4 - protein, 19 aa, Artificial Sequence
  Peptide
  MUTAGEN (1)..(1): Valeryl-Arginine
  AMIDATION (19)..(19): Arginine-amide
XPDKPRPYLPRPRPPRPVX

SEQ ID NO: 5 - protein, 19 aa, Artificial Sequence
  Peptide
  MUTAGEN (1)..(1): N,N,N′,N′-Tetramethylguanidinyl-Arginine
  AMIDATION (19)..(19): Arginine-amide
XPDKPRPYLPRPRPPRPVX

SEQ ID NO: 6 - protein, 20 aa, Artificial Sequence
  Peptide
  MUTAGEN (1)..(1): 1-amino-cyclohexyl carboxyl
XRPDKPRPYLPRPRPPRPVR

SEQ ID NO: 7 - protein, 20 aa, Artificial Sequence
  Peptide
  MUTAGEN (1)..(1): 1-amino-cyclohexyl carboxyl
  MUTAGEN (20)..(20): Arginine-hydrazide
XRPDKPRPYLPRPRPPRPVX

SEQ ID NO: 8 - protein, 20 aa, Artificial Sequence
  Peptide
  MUTAGEN (1)..(1): 1-amino-cyclohexyl carboxyl
XRPDKPRPYLPRPRPPRPVR

SEQ ID NO: 9 - protein, 5 aa, Artificial Sequence
  Peptide
  MUTAGEN (1)..(1): A variable length sequence of 1 to 10 amino acids
  MUTAGEN (5)..(5): A variable length sequence of 1 to 10 amino acids
XPRPX

SEQ ID NO: 10 - protein, 6 aa, Artificial Sequence
  PEPTIDE
  MUTAGEN (1)..(1): A variable length sequence of 1 to 10 amino acids
  MUTAGEN (6)..(6): A variable length sequence of 1 to 10 amino acids
XPRPPX

SEQ ID NO: 11 - protein, 7 aa, Artificial Sequence
  PEPTIDE
  MUTAGEN (1)..(1): A variable length sequence of 1 to 10 amino acids
  MUTAGEN (7)..(7): A variable length sequence of 1 to 10 amino acids
XPRPPRX

SEQ ID NO: 12 - protein, 8 aa, Artificial Sequence
  PEPTIDE
  MUTAGEN (1)..(1): A variable length sequence of 1 to 10 amino acids
  MUTAGEN (8)..(8): A variable length sequence of 1 to 10 amino acids
XPRPPRPX

SEQ ID NO: 13 - protein, 7 aa, Artificial Sequence
  PEPTIDE
  MUTAGEN (1)..(1): A variable length sequence of 1 to 10 amino acids
  MUTAGEN (7)..(7): A variable length sequence of 1 to 10 amino acids
XRPPRPX

SEQ ID NO: 14 - protein, 6 aa, Artificial Sequence
  PEPTIDE
  MUTAGEN (1)..(1): A variable length sequence of 1 to 10 amino acids
  MUTAGEN (6)..(6): A variable length sequence of 1 to 10 amino acids
XPPRPX

SEQ ID NO: 15 - protein, 19 aa, Artificial Sequence
  PEPTIDE
  MUTAGEN (1)..(1): Xaa can be any amino acid
  MUTAGEN (2)..(2): Xaa can be any amino acid
  MUTAGEN (4)..(4): Xaa can be any amino acid
  MUTAGEN (5)..(5): Xaa can be any amino acid
  MUTAGEN (12)..(12): Xaa can be any amino acid
  MUTAGEN (19)..(19): Xaa can be any amino acid
XXPXXPRPYLPXPRPPRPX

SEQ ID NO: 16 - protein, 20 aa, Artificial Sequence
  PEPTIDE
  MUTAGEN (1)..(1): Xaa can be any amino acid
XRPDKPRPYLPRPRPPRPVR

SEQ ID NO: 17 - protein, 19 aa, Artificial Sequence
  PEPTIDE
  MUTAGEN (1)..(1): Xaa can be any amino acid
XRPDKPRPYLPRPRPPRPV

SEQ ID NO: 18 - protein, 14 aa, Artificial Sequence
  PEPTIDE
RPYLPRPRPPRPVR

SEQ ID NO: 19 - protein, 18 aa, Artificial Sequence
  PEPTIDE
DKGSYLPRPTPPRPIYNR

SEQ ID NO: 20 - protein, 20 aa, Artificial Sequence
  PEPTIDE
  MUTAGEN (1)..(1): Xaa can be any amino acid
  MUTAGEN (2)..(2): Xaa can be any amino acid
  MUTAGEN (4)..(4): Xaa can be any amino acid
  MUTAGEN (5)..(5): Xaa can be any amino acid
  MUTAGEN (12)..(12): Xaa can be any amino acid
  MUTAGEN (19)..(19): Xaa can be any amino acid
  MUTAGEN (20)..(20): Xaa can be any amino acid
XXPXXPRPYLPXPRPPRPXX

SEQ ID NO: 21 - protein, 21 aa, Artificial Sequence
  PEPTIDE
  MUTAGEN (1)..(1): Xaa can be any amino acid
  MUTAGEN (21)..(21): Xaa can be any amino acid
XRPDKPRPYLPRPRPPRPVRX

SEQ ID NO: 22 - protein, 20 aa, Artificial Sequence
  PEPTIDE
  MUTAGEN (1)..(1): 1-amino-cyclohexyl carboxyl
XRPDKPRPYLPRPRPPRPVR

SEQ ID NO: 23 - protein, 20 aa, Artificial Sequence
  PEPTIDE
  MUTAGEN (1)..(1): 1-amino-cyclohexyl carboxyl
  AMIDATION (20)..(20): Arginine-amide
XRPDKPRPYLPRPRPPRPVX

SEQ ID NO: 24 - protein, 20 aa, Artificial Sequence
  PEPTIDE
  MUTAGEN (1)..(1): 1-amino-cyclohexyl carboxyl
  AMIDATION (20)..(20): Arginine-amide
XRPDKPRPYLPRPRPPRPVX

SEQ ID NO: 25 - protein, 41 aa, Artificial Sequence
  peptide
  MUTAGEN (1)..(1): 1-amino-cyclohexyl carboxyl
  MUTAGEN (21)..(21): 2,4-diamino-butyric acid
  MUTAGEN (41)..(41): 1-amino-cyclohexyl carboxyl
XRPDKPRPYLPRPRPPRPVRXRVPRPPRPRPLYPRPKDPRX

SEQ ID NO: 26 - protein, 41 aa, Artificial Sequence
  PEPTIDE
  MUTAGEN (1)..(1): 1-amino-cyclohexyl carboxyl
  MUTAGEN (21)..(21): 2,4-diamino-butyric acid amide
  MUTAGEN (41)..(41): 1-amino-cyclohexyl carboxyl
XRPDKPRPYLGRPRPPRPVRXRVPRPPRPRGLYPRPKDPRX